(19) United States
(12) Patent Application Publication
(10) Pub. No.: US 2023/0392195 A1
(43) Pub. Date: Dec. 7, 2023

(54) SYNTHETIC LETHALITY-MEDIATED PRECISION ONCOLOGY VIA TUMOR TRANSCRIPTOME

(71) Applicant: The United States of America, as Represented by the Secretary, Dept. of Health and Human Services, Bethesda, MD (US)

(72) Inventors: Eytan Ruppin, Bethesda, MD (US); Joo Sang Lee, Bethesda, MD (US)

(21) Appl. No.: 18/250,816

(22) PCT Filed: Oct. 29, 2021
(86) PCT No.: PCT/US21/57229

§ 371 Date: Apr. 27, 2023

Related U.S. Application Data
(60) Provisional application No. 63/107,737, filed on Oct. 30, 2020.

(51) Int. Cl. C12Q 1/6809; C12Q 1/6886

(52) U.S. Cl. CPC C12Q 1/6809; C12Q 1/6886; C12Q 2600/106

(57) ABSTRACT

The present disclosure relates to systems and methods for predicting response to cancer therapy, genes useful for predicting the sensitivity of a cancer to an anti-cancer therapy, and methods of treating such cancer.

### CROSS REFERENCE

This application claims priority to U.S. Provisional Application No. 63/107,737 entitled “SYNTHETIC LETHALITY-MEDIATED PRECISION ONCOLOGY VIA TURMOR TRANSCRPTOME” filed on Oct. 30, 2020, the entirety of which is incorporated herein by reference.

### GOVERNMENT SUPPORT

This invention was made with Government support under project number ZIA BC 011803 by the National Institutes of Health, National Cancer Institute. The United States Government has certain rights in the invention.

### FIELD OF THE DISCLOSURE

Embodiments of the present disclosure generally relate to systems and methods for predicting response to cancer therapy (either in terms of survival rates or in terms of tumor response as measured by standard Response Evaluation Criteria in Solid Tumors (RECIST) criteria) in subjects or populations affected by a disease or disorder, and more specifically for predicting components of genetic interactions, which may be used to predict the likelihood of a subject to respond to a therapy for treatment of the disease or disorder and/or predict improved therapies for treatment of the disease or disorder. The present disclosure also relates to methods of determining the sensitivity of a cancer to an anti-cancer therapy, and methods of treating such cancer.

### SUMMARY

One aspect of the present disclosure relates to a method for identifying a cancer therapy for a patient. The method may include the operations of accessing one or more databases storing information associated with genetic interactions to obtain a plurality of candidate synthetic lethality (SL) gene partners for a cancer therapy and identifying, based on experimental functional screens, patients' omics and survival data and phylogenetic profile information of each of the plurality of candidate SL gene partners, a subset of the plurality of candidate SL gene partners as potential predictive biomarkers for the cancer therapy. The method may further include the operations of comparing the subset of the plurality of candidate SL gene partners to a cancer-inhibiting drug dataset to filter the subset of the plurality of candidate SL gene partners and identifying, based on a transcriptomics profile of a tumor of the patient and the filtered subset of the plurality of candidate SL gene partners, the cancer therapy for the patient.

Another aspect of the present disclosure relates to a method for identifying a cancer therapy for a patient including accessing one or more databases storing genetic interaction information to obtain a plurality of candidate synthetic rescue (SR) gene partners for a cancer therapy and identifying, based on patients' omics and survival data and phylogenetic profile information, in particular at least one of (1) a product of PD1 and PDL1 activity (e.g., gene expression levels), (2) CTLA4 activity (e.g., protein expression levels), and (3) molecular profiles (including but limited to gene expression levels and somatic copy number alterations (SCNA)) of each of a plurality of candidate SR gene partners, a subset of candidate SR gene partners as potential predictive biomarkers for the cancer therapy. The method may also include ranking the subset of the plurality of candidate SR gene partners based on phylogenetic distance information to filter the subset of the plurality of candidate SL gene partners, filtering, based on an identification of candidate SR gene partners in which a downregulation (or upregulation) of a partner rescuer gene occurs, the ranked subset of the plurality of candidate SR gene partners, and identifying, based on a transcriptomics profile of a tumor of the patient and the filtered subset of the plurality of candidate SR gene partners, a cancer therapy for the patient.

Still another aspect of the present disclosure relates to a system for identifying a cancer therapy for a patient. The system may include a processor and a tangible storage medium storing instructions that are executed by the processor to perform the above operations.

Still another aspect of the present disclosure relates to a method of treating a cancer patient comprising administering a cancer therapy to a patient in need thereof identified according to the methods or operations described above.

Still other aspects of the present disclosure relate to assigning a score to each of the plurality of candidate SL or SR gene partners based on patient response data to each of the plurality of candidate SL or SR gene partners and filtering, based on the assigned scores, the plurality of candidate SL or SR gene partners to identify the subset of the plurality of candidate SL or SR gene partners. Ranking, based on the assigned scores, the plurality of candidate SL or SR gene partners is also contemplated, wherein the subset of the plurality of candidate SL gene partners comprises a subset of the plurality of candidate SL or SR gene partners with the highest assigned scores. Further, the subset of the plurality of candidate SL gene partners may comprise 25 gene partners (for targeted therapies) or the plurality of candidate SR gene partners may comprise 10 gene partners (for checkpoint therapy). These set size parameters and the interaction ranking schemes can be modified and improved as more datasets become available in the future. The transcriptomics profile may also include at least one of a proliferation measurement value, a cytolytic value, or a target gene expression identification level.

### BACKGROUND

There have been significant advances in precision oncology, with an increasing adoption of sequencing tests that identify targetable mutations in cancer driver genes. Aiming to complement these efforts by considering genome-wide tumor alterations at additional “-omics” layers, recent studies have begun to explore the utilization of transcriptomics data to guide cancer patients' treatment. These studies have reported encouraging results, testifying to the potential of such approaches to complement mutation panels and increase the likelihood that patients will benefit from genomics-guided, precision treatments. However, current approaches have a heuristic exploratory nature, raising the need for developing and testing new systematic approaches for utilizing tumor transcriptomics data.

One approach aims to utilize the rapidly accumulating data obtained from cancer clinical samples. One of the key objectives in this approach is to systematically map between the genomic and molecular characteristics of tumors and their responses to various drugs. One way by which to tackle this and realize the potential of cancer pharmacogenomics is based on the concept of Synthetic lethal interactions (SLi). SLi describe the relationship between two genes whereby an individual inactivation of either gene results in a viable phenotype, while their combined inactivation is lethal. SLi have been considered as a potential basis for developing selective anticancer drugs. Such drugs are aimed at inhibiting the Synthetic Lethal (SL) partner of a gene that is inactive in the cancer cells. Indeed, as 90% or more of cancer predisposing mutations result in a loss of protein function, by identifying SLi these genomic alterations can be exploited for developing and improving cancer treatments.

It is with these observations in mind, among others, that various aspects of the present disclosure were conceived and developed.

### BRIEF DESCRIPTION OF THE DRAWINGS

FIGS. 1A-1B illustrate a general method for the precision oncology framework for synthetic lethality and rescue-mediated precision oncology via the transcriptome.

FIGS. 1C-1D illustrate graphs that may be used to determine a group size and ranking of SL/SR partners.

FIGS. 1E-1J illustrate results of four melanoma cohorts treated with BRAF inhibitors identified through the operations of FIGS. 1A-1B.

FIGS. 2A-2G illustrate prediction accuracy results identified through the SL-based method of FIGS. 1A-1B on an array of different therapies and cancer types.

FIGS. 3A-3L illustrate prediction accuracy results identified through the SR-based method of FIGS. 1A-1B on an array of different therapies and cancer types.

FIGS. 4A-4K illustrate prediction accuracy results identified through the SL-based method as applied to a dataset of a multi-arm basket clinical trial setting.

FIG. 5 is a flowchart of a method for predicting survival rates in subjects or populations affected by a disease or disorder.

FIG. 6 is a diagram illustrating an example of a computing system which may be used in implementing embodiments of the present disclosure.

### DETAILED DESCRIPTION

Aspects of the present disclosure involve systems, devices, apparatus, methods, and the like, for a precision oncology framework for synthetic lethality and rescue-mediated precision oncology via the transcriptome (SELECT). The framework is generally aimed at selecting drugs or other treatments for a given patient based on the transcriptome of the patient's tumor, which may be the entire tumor transcriptome. More particularly, the presented approach is based on identifying and utilizing the broader scope of genetic interactions (GIs) of drug targets, which provide biologically testable biomarkers for therapy response prediction. Two types of GIs that are highly relevant to predicting the response to cancer therapies are considered: (1) synthetic lethal (SL) interactions, which describe the relationship between two genes whose concomitant inactivation, but not their individual inactivation, reduces cell viability (e.g., an SL interaction that is widely used in the clinic is of poly (ADP-ribose) polymerase (PARP) inhibitors on the background of disrupted DNA repair); and (2) synthetic rescue (SR) interactions, which denote a type of genetic interactions where a change in the activity of one gene reduces the cell's fitness but an alteration of another gene's activity (termed its SR partner) rescues cell viability (e.g., the rescue of Myc alterations by B-cell lymphoma 2 (BCL2) activation in lymphomas. These are relevant because when a gene is targeted by a small molecule inhibitor or an antibody, the tumor may respond by up or down regulating its rescuer gene(s), conferring resistance to therapies. The inventors have discovered that a patient's response to a cancer therapy can be predicted by analyzing SL interactions, SR interactions, or a combination thereof.

The SELECT framework comprises two basic steps: (A) For each drug whose response is to be predicted, the clinically relevant pan-cancer GIs (the interactions found to be shared across many cancer types) of the drug's target genes is identified and (B) the identified SL/SR partners of the drug emerging from step (A) is used to predict a given patient's response to a given treatment based on her/his tumor's gene expression. The operations from which SELECT differences from previous frameworks may include:
a. Generating an initial pool of SL drug target interactions for targeted therapy by following a step-wise procedure of previous frameworks while omitting certain steps not beneficial for our specific patient stratification goal. Furthermore, for the cases where no significant SL partners are found with this set procedure, introduce herein is a new procedure for relaxing the FDR thresholds in two step manner by first relaxing the FDR for the in vitro screen to 5% while keeping the FDR for tumor screen at 10%. If this relaxing does not provide any significant pairs, further relaxation of both FDRs to 20% may be performed and, if no significant pairs even with 20% FDR are not identified, the corresponding drug is declared as non-predictable by our approach;
b. Generating compact biomarker SL signatures for targeted therapy by further filtering the SL partners that pass FDR, typically ranging from 50 to 1,000 depending on the drugs and specific FDR thresholds, to generate a small set that is required to make transparent and biologically meaningful drug response predictions. The filtering of these SL partners generates a small set that is used to make transparent and biologically meaningful drug response predictions. To determine the top significant SL partners, a limited training on the BRAF inhibitor dataset is performed. Following the training, the number of top significant set size may be set to 25 where the SL partners are ranked with their survival significance;
c. Generating the initial pool of SR drug target interactions for immunotherapy by (1) for anti-PD1/PDL1 therapy, where the antibody blocks the physical interaction between PD1 and PDL1, considering the interaction term (i.e. the product of PD1 and PDL1 gene expression values) to identify the SR partners of the treatment, instead of considering just the individual expression levels of these genes, as was done in the original INCISOR pipeline. For anti-CTLA4 therapy, where the precise mechanism of action involves several ligand/receptors interactions, the CTLA4 itself may be considered, using its protein expression levels (available via reverse phase protein lysate microarray (RPPA) values in TCGA data) as they are likely to better reflect the activity than the mRNA levels. (2) Second, a step included in a previous technique aimed at identifying candidate genetic interactions from the cell line functional screening data may be omitted herein, because these interactions are not relevant to immune checkpoint response.
d. Generating compact biomarker SR signatures for immunotherapy by determining the top significant SR partners through a limited training a selected dataset such that, following the training, the number of top significant SR partners used for patient stratification may be set to 10, where the SR partners are ranked with their phylogenetic distances. The parameters may be used in making all immune checkpoint therapy response predictions. In particular, focus may be limited only on the SR interactions where inactivation of the target gene is compensated by the downregulation of the partner rescuer gene because the other types of SR interactions introduced in previous techniques may not be predictive in the training dataset.

Based on these methodological innovations, the application of SELECT to predict the response of cancer patients to a broad array of targeted and immunotherapy cancer drugs has resulted in the generation of an array of new SL and SR based biomarker signatures, for the first time. From a conceptual perspective, SELECT is shown to be the first systematic transcriptomics-based precision oncology framework that can successfully prioritize effective therapeutic options for cancer patients across many different treatments and cancer types, a much desired outcome that the previously published frameworks have fallen short of.

In one instance, transcriptomic profiles and treatment outcome information of various clinical trials may be obtained from public databases, such as a github repository. In the instances described below, information or data from a repertoire of 45 clinical trials spanning about 4,000 patients from 12 different cancer types was obtained and analyzed. In particular, cancer patient pre-treatment transcriptomics profiles may be collected together with therapy response information from numerous publicly available databases, surveying Gene Expression Omnibus (GEO), ArrayExpress and the literature, and a new unpublished cohort of anti-PD1 treatment in lung adenocarcinoma. Overall, 45 such datasets were found that includes both transcriptomics and clinical response data, spanning 12 chemotherapy, 12 targeted therapy and 21 immunotherapy datasets across 12 different cancer types.

To identify the SL and SR partners of cancer drugs, two computational pipelines may be utilized which identify genetic dependencies that are supported by multiple layers of omics data, including in vitro functional screens, patient tumor DNA and RNA sequencing data, and phylogenetic profile similarity across multiple species. The SELECT framework determines whether genetic dependencies inferred from multi-omics tumor data can be used to determine efficacious therapeutics for individual cancer patients. As such, the SELECT framework is a first of its kind systematic approach for robustly predicting clinical response to chemo, targeted and immune therapies across tens of different treatments and cancer types, offering a new way to complement existing mutation-based approaches.

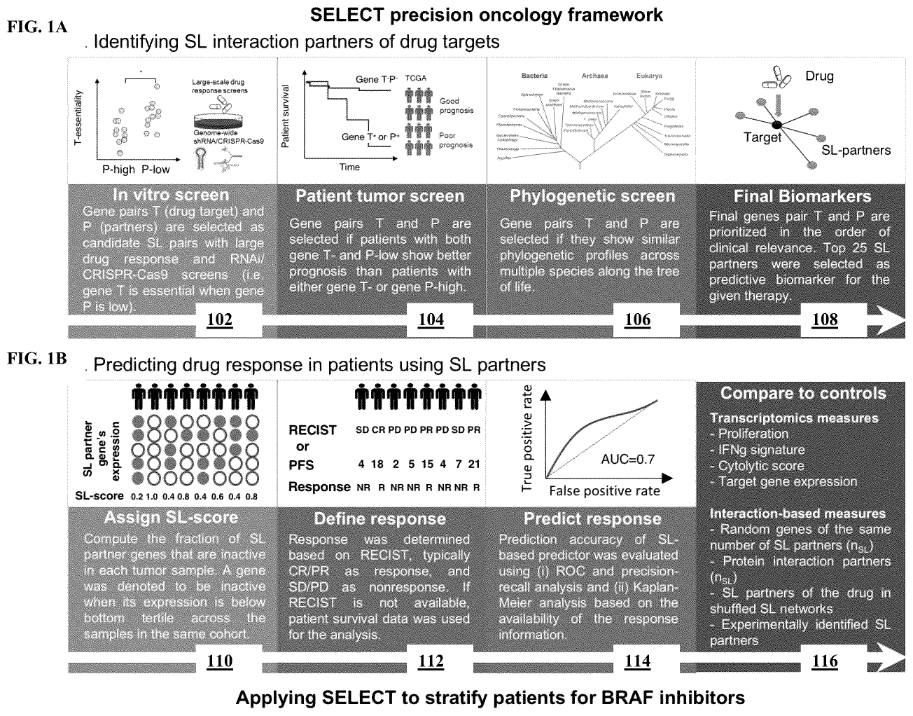

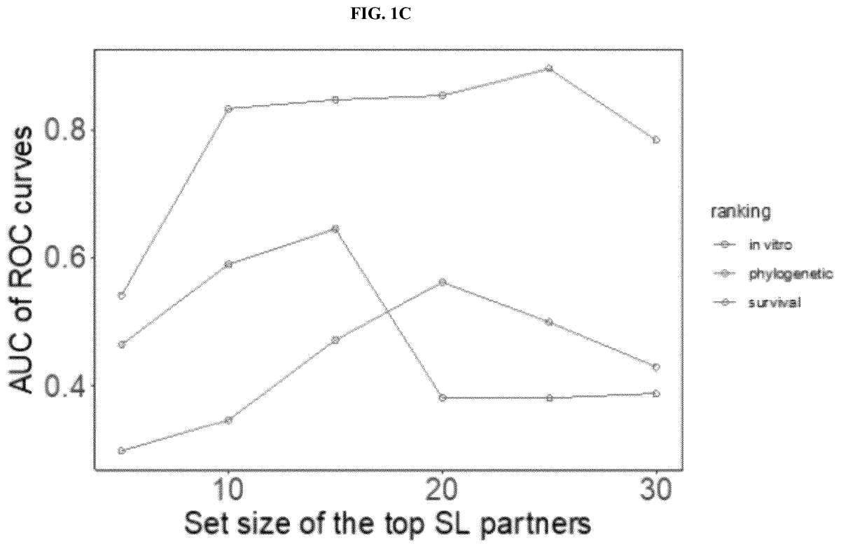

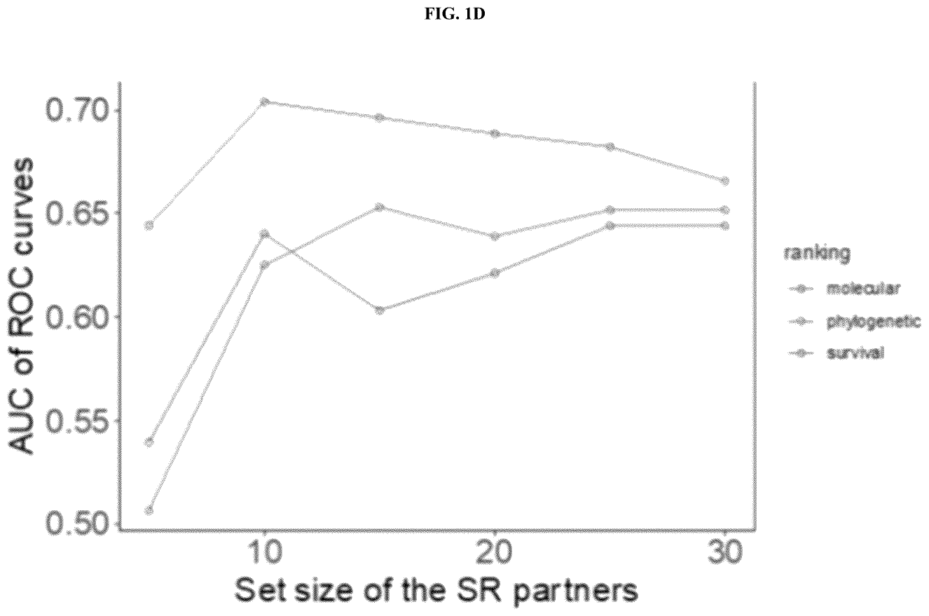

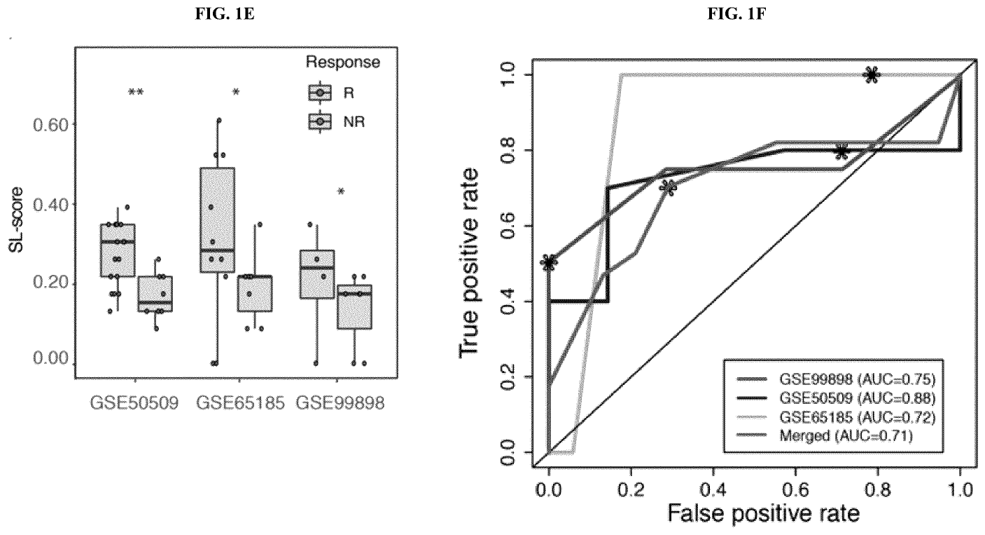

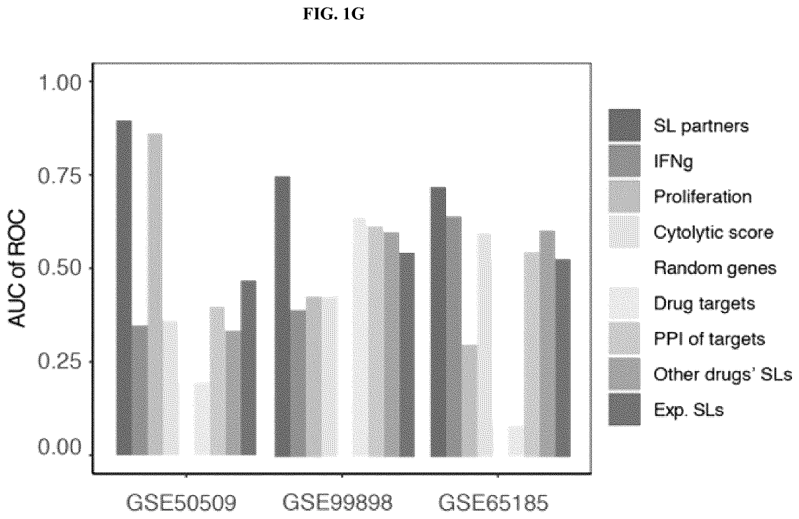

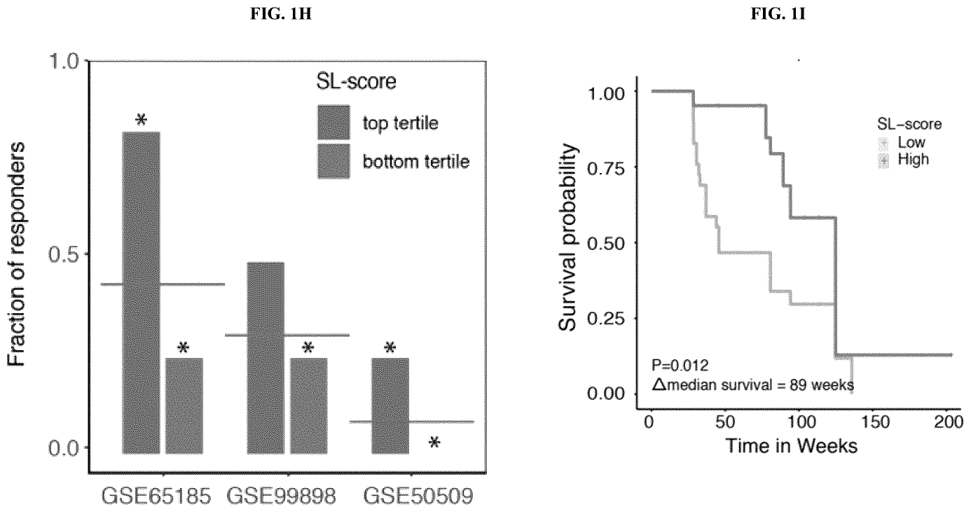

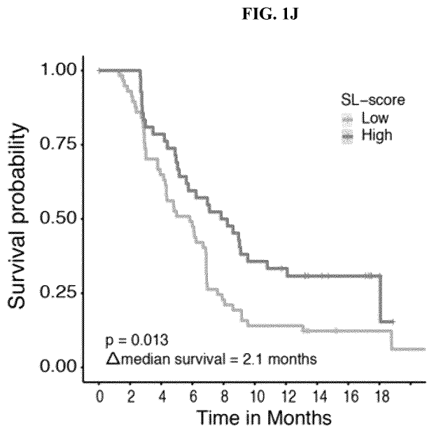

FIGS. 1A-1B illustrate a general method for the precision oncology framework for synthetic lethality and rescue-mediated precision oncology via the transcriptome. As shown, the SELECT framework includes two stages. In the first stage and for each oncology drug or therapy for which a response is to be predicted or examined, the clinically relevant pan-cancer GIs (the interactions found to be shared across many cancer types) of the drug's target genes may be identified using a computational pipeline. The identified SL partners of the drug emerging from the first stage may then be used to predict a given patient's response to a given treatment based on the patient's tumor's gene expression, the latter used to predict response to checkpoint therapy, discussed in more detail below.

FIG. 1A illustrates operations of a method for identifying and generating predictions based on SL interactions according to one implementation. Beginning in operation 102, an initial pool of SL drug target interactions for targeted therapy is generated from the obtained clinical trial data. In one instance and for each potential drug or therapy, a list of initial candidate SL pairs of its targets is compiled by analyzing large-scale in vitro functional screens performed with RNAi, CRISPR/Cas9, or pharmacological inhibition in DepMap (as outlined in Computational correction of copy number effect improves specificity of CRISPR-Cas9 essentiality screens in cancer cells, Nat Genet 49, 1779-1784 to Meyers, R. M., Bryan, J. G., McFarland, J. M., Weir, B. A., Sizemore, A. E., Xu, H., Dharia, N. V., Montgomery, P. G., Cowley, G. S., Pantel, S., et al. (2017) and Defining a Cancer Dependency Map, Cell 170, 564-576 e516 to Tsherniak, A., Vazquez, F., Montgomery, P. G., Weir, B. A., Kryukov, G., Cowley, G. S., Gill, S., Harrington, W. F., Pantel, S., Krill-Burger, J. M., et al. (2017), the contents of which are incorporated herein by reference). Among the initial list, candidate SL pairs that are more likely to be clinically relevant may be selected by analyzing the TCGA data in operation 104, looking for pairs whose downregulation is selected against and is significantly associated with better patient survival. Among the candidate pairs that remain after the two above steps, SL pairs that are supported by a phylogenetic profiling analysis may be identified and/or selected in operation 106. The most significant identified SL partners that pass all these form the pool of candidate SL partners for the specific drug. However, this may result in hundreds of significant candidate GI partners for each drug, a number which may be markedly reduced to obtain generalizable and biologically meaningful biomarker stratification signatures. The pool of candidate SL partners may then be further reduced by generating a reduced set of interaction partners to make gene therapy response predictions, by identifying optimal SL/SR set sizes and ranking criteria based on a minimal amount of supervised learning performed on one single targeted and one single immunotherapy datasets in operation 108. The number of optimal SL (and similarly for SR pairs) set size may be based on a minimal amount of supervised learning performed analyzing just one single targeted dataset. In one example, final biomarkers may be obtained from the top 25 SL partners, although any number of partners may be selected for the targeted therapy.

FIG. 1B illustrates operations for predicting drug responses in patients using SL partners obtained or selected via the method of FIG. 1A. In particular, the identified SL partners of the drug emerging from the method of FIG. 1A may be used to predict a given patient's response to a given treatment based on the gene expression profile of the individual tumor. This prediction may be based on the notion that a drug will be more effective against the tumor when its SL partners are down-regulated, because when the drug inhibits its targets more SL interactions will become jointly down-regulated and hence ‘activated’. To quantify the extent of such predicted lethality, an SL-score denoting the fraction of down-regulated SL partners of a drug in a given tumor is assigned in operation 110. Generally, the larger the fraction of SL partners being down-regulated, the higher the SL-score and the more likely the patient is predicted to respond to the given therapy. Although predictions of patient response to checkpoint therapy are based on SR pairs of the drug targets, which yield a stronger signal than their SL partners in this category of therapeutics, it should be noted that the process to infer the SR pairs of drugs and then their SR scores in each patient is analogous to that described above. In particular, the SR score of a drug in a given patient quantifies the fraction of its down-regulated SR partner genes based on the patient's tumor transcriptomics, and hence the likelihood of resistance to the given therapy.

Several of the operations described above or throughout this disclosure may include information obtained via the systems and methods described in United States Patent Application Publication No. 20190024173, entitled COMPUTER SYSTEM AND METHODS FOR HARNESSING SYNTHETIC RESCUES AND APPLICATIONS THEREOF, United States Patent Application Publication No. 20170154163, entitled CLINICALLY RELEVANT SYNTHETIC LETHALITY BASED METHOD AND SYSTEM FOR CANCER PROGNOSIS AND THERAPY, and/or United States Application Publication No. 20160300010, entitled METHOD AND SYSTEM FOR PREDICTING SELECTIVE CANCER DRUG TARGETS, the entirety of all of which are incorporated by reference herein.

In general, the SL/SR partners are inferred once analyzing DepMap and/or TCGA cohorts and their size set was optimized by training on single clinical trial dataset, prior to their application to a large collection of other test clinical trial datasets. In other words, the transcriptomic profiles and treatment outcome data available are not used in the SL and SR inference. The treatment outcomes of the selected profiles and treatment outcomes may be used to evaluate the resulting post-inference prediction accuracy in operations 112-116. Throughout the analysis, the same fixed sets of parameters in making the predictions for targeted and immunotherapies may be used. Taken together, these procedures markedly reduce the well-known risk of obtaining over-fitted predictors that would fail to predict on datasets other than those on which they were originally built.

### Generating an Initial Pool of SL Drug Target Interactions.

To identify clinically relevant SL interactions for targeted therapies, a three-step procedure may be executed, such as that disclosed in Harnessing Synthetic Lethality to Predict the Response to Cancer Treatment, Nat Commun 9, 2546 to Lee, J. S., Das, A., Jerby-Arnon, L., Arafeh, R., Auslander, N., Davidson, M., McGarry, L., James, D., Amzallag, A., Park, S. G., et al. (2018), the entirety of which is hereby incorporated by reference. The procedure may include (1) creating an initial pool of SL pairs identified in cell lines via RNAi/CRISPR-Cas9 (as outlined in Meyers et al., 2017 and Tsherniak et al. (2017) or pharmacological screens (as outlined in An Interactive Resource to Identify Cancer Genetic and Lineage Dependencies Targeted by Small Molecules, Cell 154, 1151-1161 to Basu, A., Bodycombe, N. E., Cheah, J. H., Price, E. V., Liu, K., Schaefer, G. I., Ebright, R. Y., Stewart, M. L., Ito, D., Wang, S., et al. (2013), the contents of which are incorporated herein by reference. For drug target gene T and candidate SL partner gene P, growth reduction induced by knocking out/down gene T or pharmacologically inhibiting gene T is stronger when gene P is inactive is checked, via a Wilcoxon ranksum test. (2) Second, among the candidate gene pairs from the first step, gene pairs are selected whose co-inactivation is associated with better prognosis in patients, using a Cox proportional hazard model, testifying that they may thus hamper tumor progression. (3) SL paired genes with similar phylogenetic profiles across different species may be prioritized. Because of the distinct distribution of P-values for the first two screens, a false discovery correction may be performed with 1% for the in vitro screen (1^{st} step) and 10% for the tumor screen (2^{nd} step).

Through these operations, identification of significant SL partners with these False Discovery Rate (FDR) thresholds for most of the datasets is made. However, for the cases in which significant SL partners with this set of FDR thresholds are not found, the FDR thresholds may be identified in a two step manner; by relaxing the FDR for the in vitro screen to 5% while keeping the FDR for tumor screen at 10%, or further relaxing both FDRs to 20%. If no significant pairs are identified even with 20% FDR, the corresponding drug may be identified as non-predictable by the instant approach.

### Generating a Subset of SL Signatures.

The number of SL partners that pass FDR ranges from 50 to 1,000 may depend on the drugs and specific FDR thresholds. Accordingly, SL partners may be filtered to generate a small set that is used to make the drug response predictions. This further filtering has been motivated by the following three reasons: (1) Occam's razor (regularization): predictor with a smaller number of variables are likely to generalize better; (2) biomarker interpretability: small sets of partners are more relevant for clinical use as predictive biomarkers; and (3) patient cohort analysis: when comparing the SL-scores of different drugs to decide which would be a best fit for a given patient, using the same number of top predictors facilitates such an analysis on equal grounds. To determine the top significant SL partners, a limited training on a data set may be conducted. In one example, the data set is the BRAF inhibitor dataset (GSE50509). For example, FIG. 1C illustrates a graph that may be used to determine a group size and ranking of SL partners. More particularly, shown is the top significant SL partners used in the prediction of cytotoxic/targeted agents and immunotherapy through variation of sizes and rankings in the relevant datasets. The graph illustrates the resulting prediction performance on the selected datasets. Following the training, the number of top significant set size may be set to 25 where the SL partners are ranked with their survival significance. From the graph of FIG. 1C, 25 SL partners may be selected as the SL partners set size and survival p-values as the ranking scheme used in the analysis of all other cytotoxic/targeted agents.

### Generating an Initial Pool of SR Drug Target Interactions.

To identify GIs for immunotherapy, a general GI inference pipeline may be altered to incorporate the characteristics of immune checkpoint therapy (as disclosed in Lee et al., 2018 and Genome-wide prediction of synthetic rescue mediators of resistance to targeted and immunotherapy, Mol Syst Biol 15, e8323 to Sahu, A. D., J, S. L., Wang, Z., Zhang, G., Iglesias-Bartolome, R., Tian, T., Wei, Z., Miao, B., Nair, N. U., Ponomarova, O., et al. (2019)). In one example, for anti-PD1/PDL1 therapy, where the antibody blocks the physical interaction between PD1 and PDL1, the interaction term (i.e. the product of PD1 and PDL1 gene expression values) may be considered to identify the SR partners of the treatment. For anti-PD1/PDL1 therapy, gene expression may be utilized or analyzed, rather than protein expression, as protein expression of PD1 and PDL1 may not be available for many samples. In another example, for anti-CTLA4 therapy, where the precise mechanism of action involves several ligand/receptors interactions, the CTLA4 itself may be considered, using its protein expression levels (available via reverse phase protein lysate microarray (RPPA) values in TCGA data) as they are likely to better reflect the activity than the mRNA levels. (2) For the GI partner levels, gene expression and somatic copy number alterations (SCNA) data may be utilized as protein expression may be measured only for a small subset of genes. (3) Instead of considering all protein coding genes as candidates for SR partners, the genes that are covered by the NanoString panel may be considered because (i) the gene expression of many of ICI datasets was quantified by NanoString platform and (ii) NanoString panel is enriched with immune system related genes that are highly relevant to the response to immune checkpoint therapy. (4) The first step of the SL/SR inference procedure, which is aimed at identifying candidate genetic interactions from the cell line functional screening data, may be omitted because these interactions are not relevant to immune checkpoint response. In some instances, the genome-wide CRISPR screens in cancer cell/T-cell co-culture may be used, but this data is limited to melanoma and the coverage is not fully genome-wide, where many genes included in the NanoString panel are missing. (5) The mediators of resistance to immune checkpoint therapies using synthetic rescue (SR) interactions, as no statistically significant SL interaction partners may be identified for either PD1 or CTLA4, may also be used. False discovery correction was done with FDR 10%.

### Generating Subset of SR Signatures

To determine the top significant SR partners, a limited training may be conducted on a dataset, as illustrated in the graph FIG. 1D. In particular, the graph illustrates a graph that may be used to determine a group size and ranking of SR partners. More particularly, shown is the top significant SR partners used in the prediction of targeted immunotherapy through variation of sizes and rankings in the Van Allen dataset disclosed in Genomic correlates of response to CTLA-4 blockade in metastatic melanoma, Science 350, 207-211 to Van Allen, E. M., Miao, D., Schilling, B., Shukla, S. A., Blank, C., Zimmer, L., Sucker, A., Hillen, U., Foppen, M. H. G., Goldinger, S. M., et al. (2015). The graph illustrates the resulting prediction performance on the selected dataset. Following the training, the number of top significant set size may be set to 10 where the SR partners are ranked with their phylogenetic distances. These parameters may be used in making all immune checkpoint therapy response predictions. TCGA data applying this pipeline may be analyzed to identify pan cancer SR interactions that are more likely to be clinically relevant across many cancer types. In particular, the SR interactions where inactivation of the target gene is compensated by the downregulation (or upregulation) of the partner rescuer gene may be highlighted, as the other types of SR interactions introduced in were not predictive in the training dataset.

### Predicting Drugs Response Based on SL/SR Partners

To predict drug response in patients using SL/SR partners, one or more of the identified SL or SR partners for drug response prediction may be analyzed. In particular, an SL-score for chemotherapy and targeted therapy may be defined as the fraction of inactive SL partners in a given sample out of all SL partners of that drug following the notion that an inhibitor would be more effective when a larger number of its drug target genes' SL partners are inactive. The SL score reflects the intuitive notion that inhibiting targeted drug would be more effective when a larger fraction of its SL partners is inactive in the tumor. In each patient drug response dataset, a gene is determined to be inactive if its expression is below bottom tertile across samples in the same dataset. This normalization may be performed (i) to account for the basal expression level of each gene in specific tumor type and (ii) to minimize batch effect occurring when different datasets are combined. Additionally, the SL-score may be multiplied by a target gene factor to obtain the final SL score. This has been motivated by the notion that an inhibitor will be not effective when its target gene is not expressed; thus, the target gene factor may be set to be zero when the target gene is inactive (below bottom 30-percentile in the given sample), and mean expression of the targets genes may be used when the given drug has more than one target gene. SR-scores may be used to predict response to immunotherapy, which quantifies the fraction f, SR partners that are inactive, and 1-f as the SR-score to predict responders. Higher SL- or SR-score is generally predictive of response to therapies.

Using the computed SL/SR-scores, either the classification problem to predict responders may be solved or Kaplan-Meier analysis may be performed to predict patient survival, depending on the availability of the data. For the datasets where the response information is available in the form of RECIST criteria, solving the classification problem may be performed. For the cases where progression-free survival time is available for all patients (with no censoring event), the median progression-free survival from the relevant literature as the cutoff to distinguish the responders from non-responders may be used to solve a classification problem. For the datasets where only overall or progression-free survival with censoring information are available, Kaplan-Meier analysis may be performed.

In still other instances, TCGA anti-PD1 coverage analysis for predicting the cancer type-specific response to checkpoint therapy may be performed. The objective response rates of anti-PD1 therapy in each cancer type in TCGA may thus be predicted via the SR interaction partners of PD1 identified above. The SR scores in each tumor sample in the TCGA compendium may be computed, based on its transcriptomics profiles following the above definition of SR-score, and labeled it as responder or non-responder accordingly using the point of maximal F1-score as threshold across all 9 immune checkpoint datasets, where the SR-score is predictive. Using this fixed cut-off, the fractions of responders for each cancer type may be computed and compared with the actual response rates reported in anti-PD1 clinical trials for 16 cancer types where the data is available using Spearman rank correlation. In each patient drug response datasets, a gene is determined to be inactive if its expression is below bottom tertile across samples in the same dataset following the previous studies.

### Methods of Determining Cancer Sensitivity to Anti-Cancer Therapies and Treating Cancer

Provided herein are methods of determining the susceptibility and/or sensitivity of a cancer to a particular anti-cancer therapy, and applications thereof for treating a cancer in a subject. The subject may be a human patient in need of anti-cancer therapy. The method may comprise determining the SL-score of a subject's cancer sample for the anti-cancer therapy, which may be indicative of the sensitivity of the subject's cancer to the anti-cancer therapy. The method may also comprise administering the anti-cancer therapy to the subject based on the SL-score for the anti-cancer therapy. In one example, a SL-score >0.44 indicates that the subject's cancer is sensitive to the anti-cancer therapy.

An anti-cancer therapy may comprise a drug or drug combination listed in Table 1, and the SL partner genes indicative of the sensitivity of the subject's cancer to the anti-cancer therapy may comprise the group of genes listed in Table 1 that are associated with the anti-cancer therapy. The SL partner genes for an anti-cancer therapy used to determine the SL-score of a subject's cancer may also consist of the SL partner genes listed in Table 1. In one example, the anti-cancer therapy is Vemurafenib, and the SL partner genes comprise FSCN1, ICA1, PMS2, C1GALT1, MMD2, C7orf28B, NT5C3, NDUFA4, RAPGEF5, TMEM106B, ADCYAP1R1, SCIN, NEUROD6, RP9, FAM126A, KLHL7, SKAP2, TRA2A, JAZF1, CBX3, BBS9, SP8, MACC1, GGCT, and TAX1BP1A. In another example, the anti-cancer therapy is Tamoxifen, and the SL partner genes comprise LTBP2, GADL1, CRISP2, SLC13A5, PCDHGA7, NLRP10, AAK1, IL22RA2, RASGRF1, FAM19A3, TPM2, UBR4, LRRFIP1, FOXL1, PCDHGA2, MAMSTR, ABCG4, FBXO32, DSG3, FER, ALPP, PINX1, AVPR1A, LHX6, and PHLPP2. Other examples are provided in Table 1.

Also provided herein are methods of determining the susceptibility and/or sensitivity of a cancer when the anti-cancer therapy is a checkpoint therapy, and applications thereof for treating a cancer in a subject. The subject may be a human patient in need of checkpoint therapy. The method may comprise determining the SR-score of a subject's cancer sample for the checkpoint therapy, which may be indicative of the sensitivity of the subject's cancer to the checkpoint therapy. The method may also comprise administering the checkpoint therapy to the subject based on the SR-score for the checkpoint therapy. In one example, a SR-score ≥0.9 indicates that the subject's cancer is sensitive to the checkpoint therapy.

The checkpoint therapy may be a PD1/PDL1 inhibitor or an anti-CTLA4 therapy. The PD1/PDL1 inhibitor may any such inhibitor known in the art, and may be Pembrolizumab, Nivolumab, Cemiplimab, Atezolizumab, Avelumab, or Durvalumab. For the PD1/PDL1 inhibitor, the SR partner genes used to determine the SR-score may comprise CXCL16, IL15RA, CD27, TNFRSF13C, TNFRSF13B, ICAM4, CD8A, CD4, LTBR, and IFITM2. The anti-CTLA therapy may be any such therapy known in the art, and may be Ipilimumab or tremelimumab. For the anti-CTLA4 therapy, the SR partner genes used to determine the SR-score may comprise CD44, IL22RA2, THBD, BID, F12, CCL13, EWSR1, CD274, IL22RA1, and CDKN1A.

Some of the types of cancers that can be treated by chemo-, targeted- or immuno-checkpoint therapies disclosed herein are known in the art, but a subject's cancer may be sensitive to an anti-cancer or checkpoint therapy even if the therapy has not received regulatory approval for treating the cancer, or has not previously been recognized as being effective against the type of cancer the subject has. Thus, a SL- or SR-score for the subject's cancer may be useful for identifying new types of cancers that are sensitive to the anti-cancer therapy or checkpoint therapy.

The SL- or SR-score may be determined according to a method described herein. In one example, for a given anti-cancer therapy, expression levels of the SL or SR partner genes may be provided from a sample of the subject's cancer, and from each of a plurality of reference cancer samples. The number of the SL or SR partner genes that are downregulated in the subject's cancer sample as compared to expression levels in the reference cancer samples may be counted. In one example, a SL or SR partner gene expressed in the subject's cancer sample may be downregulated if its expression levels are in the bottom half, tertile, quartile, or quintile of expression levels of that SL or SR partner gene as measured among the reference cancer samples. In one example, a SL or SR partner gene is downregulated in the subject's cancer sample if the expression level of the SL or SR partner gene is in the bottom tertile of expression levels of the SL or SR partner gene among the reference cancer samples.

To determine a SL-score for an anti-cancer therapy, the number of the SL partner genes that are downregulated in the subject's cancer sample may be divided by the total number of SL partner genes associated with the anti-cancer therapy. In one example, a SL-score >0.44 (for example, where at least 11 of 25 SL partner genes are downregulated in the subject's cancer sample as compared to the reference cancer samples) indicates that the subject's cancer is sensitive to the anti-cancer therapy.

To determine a SR-score for a checkpoint therapy, the number of the SR partner genes that are downregulated in the subject's cancer sample may be divided by the total number of SR partner genes associated with the checkpoint therapy. The result of that calculation may then be subtracted from 1 to determine the SR-score. In one example, a SR-score indicates that the subject's cancer is sensitive to the checkpoint therapy.

A cancer sample referred to herein may be any type of sample known in the art, but may in particular comprise a bulk tumor biopsy. The reference cancer samples may be of the same type of cancer as the subject's cancer. If the subject's cancer type is unknown, then the reference cancer samples may comprise one or more types of cancer that are different from the subject's, and in one example may comprise all cancer samples from a source of SL or SR partner gene expression levels.

The SL or SR partner gene expression levels may be measured from RNA-sequencing (RNAseq) or a microarray data. The gene expression levels may be normalized. In one example, the same normalization method may be used for SL or SR partner gene expression levels of the subject's cancer sample and of the reference cancer samples. The normalization method may be Reads per Kilobase per Million mapped reads (RPKM)/RNAseq by Expectation-Maximization (RSEM), which may be particularly useful when gene expression levels are measured using RNAseq. The SL or SR partner gene expression levels of the reference cancer samples may be provided from any source of data known in the art. The data source may be a database, and may be the Cancer Genome Atlas (TCGA), which is available at www.cancer.gov/tcga (the contents of which are incorporated herein by reference). The cancer may be any cancer known in the art, and may be one described in the TCGA.

TABLE 1
Anti-Cancer Therapies and Associated SL Partner Genes

| Drug | SL partner genes |
|---|---|
| Vemurafenib | FSCN1 |
| | ICA1 |
| | PMS2 |
| | C1GALT1 |
| | MMD2 |
| | C7orf28B |
| | NT5C3 |
| | NDUFA4 |
| | RAPGEF5 |
| | TMEM106B |
| | ADCYAP1R1 |
| | SCIN |
| | NEUROD6 |
| | RP9 |
| | FAM126A |
| | KLHL7 |
| | SKAP2 |
| | TRA2A |
| | JAZF1 |
| | CBX3 |
| | BBS9 |
| | SP8 |
| | MACC1 |
| | GGCT |
| | TAX1BP1 |
| Tamoxifen | LTBP2 |
| | GADL1 |
| | CRISP2 |
| | SLC13A5 |
| | PCDHGA7 |
| | NLRP10 |
| | AAK1 |
| | IL22RA2 |
| | RASGRF1 |
| | FAM19A3 |
| | TPM2 |
| | UBR4 |
| | LRRFIP1 |
| | FOXL1 |
| | PCDHGA2 |
| | MAMSTR |
| | ABCG4 |
| | FBXO32 |
| | DSG3 |
| | FER |
| | ALPP |
| | PINX1 |
| | AVPR1A |
| | LHX6 |
| | PHLPP2 |
| Anthracycline | NFKB1 |
| | ZNF667 |
| | NMNAT1 |
| | DAPK1 |
| | ELOVL7 |
| | TINAGL1 |
| | PCDHGA7 |
| | ZNF470 |
| | PIWIL4 |
| | ZNF471 |
| | ZNF300 |
| | GALNTL2 |
| | CPM |
| | ECHDC3 |
| | RBM7 |
| | POU2F2 |
| | ARHGAP6 |
| | H6PD |
| | EIF4G3 |
| | NCF4 |
| | SH3GLB1 |
| | AADAC |
| | SLC25A24 |
| | STX11 |
| | ADAMTS5 |
| Lapatinib/Epirubicin/Fluorouracil | SLC7A6 |
| | NFKB1 |
| | ZNF667 |
| | ELOVL7 |
| | TINAGL1 |
| | LTBP4 |
| | AP4E1 |
| | PCDHGA7 |
| | PIWIL4 |
| | KIF3C |
| | ZNF471 |
| | AADACL2 |
| | CPM |
| | ECHDC3 |
| | RBM7 |
| | POU2F2 |
| | STXBP1 |
| | RPL4 |
| | TOMM40L |
| | H6PD |
| | ALS2 |
| | AMMECR1L |
| | PACS1 |
| | CSMD3 |
| | RLBP1 |
| Anastrozole | MMP21 |
| | ZNF662 |
| | ANKRD1 |
| | ALPP |
| | H2AFY2 |
| | FGF10 |
| | DCAF12L1 |
| | KIAA1549 |
| | RBM12 |
| | PTPRN2 |
| | LRRC8A |
| | TSPAN14 |
| | MYH11 |
| | SLC4A7 |
| | HECW2 |
| | NKD1 |
| | ARHGDIG |
| | RC3H2 |
| | MLL3 |
| | DRD1 |
| | TAF1L |
| | SLC7A6 |
| | ZXDB |
| | VSIG2 |
| | TMEFF2 |
| Bortezomib/Thalidomide/Dexamethasone | ALPP |
| | LCTL |
| | LTA4H |
| | BMP8B |
| | CPN2 |
| | NAALAD2 |
| | KRT16 |
| | ZNF600 |
| | IGF2BP3 |
| | RPP25 |
| | ACOT8 |
| | TNNC1 |
| | CGB |
| | PTGES |
| | SLC17A7 |
| | ROS1 |
| | EPHA8 |
| | ABCG4 |
| | CYP11A1 |
| | PKP3 |
| | MYL6 |
| | STAU1 |
| | F3 |
| | LTBP4 |
| | FGF23 |
| Irinotecan/Fluorouracil | MTRF1L |
| | MAPK11 |
| | TXK |
| | RPL4 |
| | NADK |
| | SLC7A6 |
| | ACOX2 |
| | SOD2 |
| | STXBP1 |
| | SLC9A8 |
| | LIMK2 |
| | ARHGDIG |
| | DIRAS1 |
| | SLC2A5 |
| | DUSP7 |
| | RPL5 |
| | STK32A |
| | CCND3 |
| | SOD2 |
| | ECHDC3 |
| | CSNK1A1 |
| | PCSK7 |
| | CABP4 |
| | KIF1B |
| | LDHAL6B |
| Doxorubicin/Gemcitabine | NFKB1 |
| | ZNF667 |
| | NMNAT1 |
| | DAPK1 |
| | ELOVL7 |
| | SMAP2 |
| | TINAGL1 |
| | PCDHGA7 |
| | ZNF470 |
| | PIWIL4 |
| | ZNF471 |
| | ERI1 |
| | KCNH4 |
| | AADACL2 |
| | RPL11 |
| | RFX2 |
| | CPM |
| | ECHDC3 |
| | RBM7 |
| | POU2F2 |
| | STXBP1 |
| | AGTPBP1 |
| | SLC6A16 |
| | CFI |
| | ARHGAP6 |
| Folinic Acid (FA)/Irinotecan/Fluorouracil | FAM83G |
| | DNAJC12 |
| | RALBP1 |
| | TXK |
| | RPL4 |
| | NADK |
| | MYBPC2 |
| | AGTR2 |
| | SLC7A6 |
| | ACOX2 |
| | SOD2 |
| | STXBP1 |
| | LIMK2 |
| | ARHGDIG |
| | DIRAS1 |
| | POLA2 |
| | SLC2A5 |
| | SLC9A9 |
| | PMP22 |
| | SOD2 |
| | ECHDC3 |
| | KIRREL |
| | NTAN1 |
| | CABP4 |
| | KIF1B |
| Afatinib | CHCHD3 |
| | SLC7A6 |
| | EPHB2 |
| | CFL1 |
| | SLCO4A1 |
| | PAX3 |
| | MORC2 |
| | NR0B1 |
| | OR1F1 |
| | RADIL |
| | KCNH2 |
| | LTBP4 |
| | ADCY3 |
| | KIF3C |
| | TMSB10 |
| | STYXL1 |
| | TAGLN2 |
| | NFKBIL2 |
| | PUSL1 |
| | JAM3 |
| | TRIP13 |
| | PTDSS1 |
| | QSOX1 |
| | CDCA7L |
| | APCDD1L |
| Azacitidine | PCDHGA7 |
| | MLLT4 |
| | CHD5 |
| | WFIKKN2 |
| | DRD1 |
| | BACH1 |
| | TUB |
| | PDZD4 |
| | CLDN18 |
| | PCDHGA10 |
| | PCDHGA6 |
| | BAZ2A |
| | KIAA0355 |
| | ESYT2 |
| | PRSS33 |
| | ZNF300 |
| | CAPS2 |
| | RHOBTB3 |
| | PCDHGA5 |
| | KDM6B |
| | PRDM2 |
| | ZNF573 |
| | GPR114 |
| | AADAC |
| | ANKRD17 |
| Belinostat | SOD2 |
| | TTPAL |
| | VPS26A |
| | AURKC |
| | KLF17 |
| | CYTH3 |
| | EIF1 |
| | CTH |
| | TSC22D2 |
| | RER1 |
| | AGPAT4 |
| | ATP5B |
| | DACT1 |
| | RNF24 |
| | HIST1H2AL |
| | CEBPB |
| | MFI2 |
| | DDX19B |
| | VAMP3 |
| | GNB1 |
| | PGK2 |
| | GFRA2 |
| | NR5A1 |
| | RNF24 |
| | MAN2A1 |
| Bortezomib | LTA4H |
| | TNNC1 |
| | MYL6 |
| | RPS12 |
| | MYL6 |
| | PROSC |
| | ORMDL2 |
| | CAPZB |
| | VAMP3 |
| | RPS8 |
| | SOD2 |
| | PGM1 |
| | GPX7 |
| | PRDX6 |
| | DDX47 |
| | CSNK2A2 |
| | PGM1 |
| | SFN |
| | CPS1 |
| | KPNA6 |
| | KARS |
| | AKT2 |
| | MRTO4 |
| | EIF3I |
| | ARL4C |
| Cabozantinib | TOMM40L |
| | LAMB4 |
| | NUCB1 |
| | PCDHB13 |
| | SERTAD1 |
| | FBXL22 |
| | C2orf24 |
| | INPP5A |
| | C14orf138 |
| | USP35 |
| | BLCAP |
| | NIPSNAP3A |
| | TACR2 |
| | ZNF257 |
| | EIF2B3 |
| | WNT10A |
| | ZYX |
| | PCDH8 |
| | KDM4B |
| | KCNJ14 |
| | LGALS7 |
| | AVEN |
| | TMEM18 |
| | TNC |
| | NRBF2 |
| Carfilzomib | RPS12 |
| | SRF |
| | BIN3 |
| | PSMD7 |
| | PINX1 |
| | MSRA |
| | FDFT1 |
| | ERI1 |
| | LONRF1 |
| | PCDHAC1 |
| | SOX7 |
| | UBA7 |
| | TLE4 |
| | PPP1R3B |
| | MTMR9 |
| | TMEM110 |
| | GATA4 |
| | SLC7A2 |
| | RAB27A |
| | NEFM |
| | CTSB |
| | PRPF38A |
| | KCNAB2 |
| | PAQR5 |
| | MBD3L1 |
| Cetuximab | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Cobimetinib | CPT2 |
| | RPL5 |
| | CTDSPL2 |
| | MAP1LC3A |
| | HBS1L |
| | USP3 |
| | MRTO4 |
| | ELOVL6 |
| | PRKACG |
| | TADA2A |
| | CUL5 |
| | ME3 |
| | STXBP3 |
| | DHX35 |
| | CSNK1G3 |
| | RBBP4 |
| | MRE11A |
| | NAA16 |
| | BCAT2 |
| | RAB33B |
| | DDX46 |
| | CABP5 |
| | WDR26 |
| | LACTB2 |
| | PRPSAP1 |
| Crizotinib | TOMM40L |
| | PCDHB13 |
| | FBXL22 |
| | C2orf24 |
| | C1GALT1 |
| | INPP5A |
| | USP35 |
| | TACR2 |
| | ZNF257 |
| | KDM4B |
| | TMEM18 |
| | ACOX2 |
| | RXRG |
| | BMP8B |
| | AZU1 |
| | FAM36A |
| | NAT15 |
| | PLA2G3 |
| | C10orf140 |
| | PHYH |
| | SS18 |
| | BRMS1L |
| | C17orf61 |
| | PHTF1 |
| | HNRNPH3 |
| Denileukin | PSAPL1 |
| | CNTF |
| | MTL5 |
| | ART5 |
| | ZNF300 |
| | TACR2 |
| | EPHX3 |
| | WBP11P1 |
| | CCK |
| | SKP2 |
| | EXD1 |
| | NFE2L3 |
| | B3GALT5 |
| | NEU4 |
| | FAM3B |
| | NPPC |
| | KLHL34 |
| | HUWE1 |
| | TREML2 |
| | GAL |
| | C12orf48 |
| | C2orf43 |
| | ZNF829 |
| | HBQ1 |
| | FAM105B |
| Dinutuximab | ZIK1 |
| | RFX2 |
| | RNF24 |
| | CDKL5 |
| | FAM179B |
| | NCOA3 |
| | GPRC5C |
| | SPINK2 |
| | DNAJB12 |
| | S100B |
| | CIDEA |
| | CYP11A1 |
| | TBC1D2 |
| | CLMN |
| | SLC16A12 |
| | NPTX2 |
| | SHANK2 |
| | CEP164 |
| | DAAM1 |
| | MMP15 |
| | MAST4 |
| | MAPK4 |
| | GPRIN2 |
| | MT1A |
| | IL18 |
| Durvalumab | HMGA1 |
| | E2F2 |
| | TMEM69 |
| | C20orf20 |
| | PHLDA2 |
| | BIRC7 |
| | PSAPL1 |
| | C1orf135 |
| | POLQ |
| | IQGAP3 |
| | PRAME |
| | KIAA1524 |
| | ZNF598 |
| | ZNF695 |
| | ZNF581 |
| | HTATIP2 |
| | SLCO4A1 |
| | TMPRSS13 |
| | KIF23 |
| | LMNB2 |
| | CCNA2 |
| | BUB1 |
| | PSRC1 |
| | SLC16A1 |
| | KIFC1 |
| Estramustine | MEA1 |
| | PKHD1 |
| | MRPS10 |
| | BMP8B |
| | SSB |
| | LMLN |
| | ZNF829 |
| | HSD11B1 |
| | STXBP5 |
| | ZNF239 |
| | NCDN |
| | GLIS2 |
| | QSOX1 |
| | RBMS3 |
| | KRT75 |
| | MASTL |
| | ITGA11 |
| | C1QC |
| | TMC7 |
| | MS4A8B |
| | ADCY7 |
| | FGF5 |
| | MLF1 |
| | KIF4B |
| | PLP2 |
| Everolimus | PRKCG |
| | DNAJB12 |
| | CDK16 |
| | C20orf118 |
| | STK4 |
| | CTH |
| | NOX1 |
| | DNAJC2 |
| | FGR |
| | TAF12 |
| | DDOST |
| | RPS6KA1 |
| | TSSK3 |
| | RHEBL1 |
| | SLC7A6 |
| | MYLK4 |
| | KMO |
| | PSMB9 |
| | HERC4 |
| | SGPL1 |
| | PTPN6 |
| | SLC2A5 |
| | NOL10 |
| | TRNAU1AP |
| | VAC14 |
| Gemtuzumab | WNT10B |
| | TAF4 |
| | MFSD2B |
| | PTGDS |
| | SPRR3 |
| | GATAD2A |
| | OPRL1 |
| | ALOX15 |
| | MESP2 |
| | PSAPL1 |
| | B3GALT5 |
| | LRRC42 |
| | BIRC7 |
| | SLC4A2 |
| | PLEKHA6 |
| | NR5A1 |
| | SOX10 |
| | HNRNPL |
| | KLHL30 |
| | GALK1 |
| | RHPN2 |
| | STAU1 |
| | DBNDD1 |
| | UPK1A |
| | NCDN |
| Ibrutinib | SLC6A14 |
| | CFL1 |
| | ABCC10 |
| | CORO1B |
| | OPRL1 |
| | HORMAD1 |
| | RCE1 |
| | ZNF239 |
| | ZDHHC7 |
| | GPRC5D |
| | KRT78 |
| | KRT6C |
| | RGS1 |
| | PADI3 |
| | UNC13D |
| | B3GALT5 |
| | WNT10B |
| | C1GALT1 |
| | HSD17B2 |
| | RGS19 |
| | MARK2 |
| | NAA40 |
| | SLC7A6 |
| | DBNL |
| | UPP1 |
| Idelalisib | MFSD2B |
| | POLQ |
| | TXK |
| | CELA3B |
| | ZC3H12A |
| | GNL3 |
| | ATAD5 |
| | DNAJC2 |
| | LARP1 |
| | C1GALT1 |
| | TAS1R1 |
| | RPRD1B |
| | HDLBP |
| | KRT75 |
| | STK4 |
| | CCDC19 |
| | NOL10 |
| | BHLHE40 |
| | RNF24 |
| | CNNM4 |
| | SLC7A5 |
| | CLSPN |
| | CREG2 |
| | LRRC42 |
| | MYC |
| Lapatinib | SLC7A6 |
| | B4GALT5 |
| | PACS1 |
| | CSMD3 |
| | VPS13B |
| | CHRFAM7A |
| | PGCP |
| | MTBP |
| | RIMS2 |
| | TMEM74 |
| | NIPAL2 |
| | C8orf37 |
| | RNF19A |
| | ADCY3 |
| | FBXO43 |
| | SNX31 |
| | KCNS2 |
| | HAS2 |
| | KIAA0196 |
| | STX16 |
| | DOK5 |
| | UTP23 |
| | CDH17 |
| | DERL1 |
| | TTPAL |
| Lenvatinib | PIGT |
| | KNTC1 |
| | SNX5 |
| | LEP |
| | TAX1BP3 |
| | FAM83D |
| | ERGIC1 |
| | FGF5 |
| | LRRC42 |
| | MYC |
| | KCNA7 |
| | HOXC8 |
| | CDC25B |
| | P4HA3 |
| | KIF3C |
| | GREM1 |
| | CDC25B |
| | CDC25B |
| | LAMB1 |
| | FLNC |
| | FHOD1 |
| | SLC7A6 |
| | ZFHX4 |
| | ITGA5 |
| | NUMBL |
| Midostaurin | FLNC |
| | COTL1 |
| | CPA4 |
| | GREM1 |
| | HOXC5 |
| | IGF2BP1 |
| | IGF2BP3 |
| | PDLIM7 |
| | SKP2 |
| | HOXC8 |
| | YBX1 |
| | HNRNPL |
| | DPH2 |
| | C15orf42 |
| | EPHB2 |
| | HTR1D |
| | ZDHHC7 |
| | NADK |
| | CTHRC1 |
| | DHX34 |
| | CPXM1 |
| | TPX2 |
| | DCLRE1B |
| | CDCA7 |
| | PDLIM3 |
| Nintedanib | SND1 |
| | PIGT |
| | CHD5 |
| | PLXNA3 |
| | SEH1L |
| | HNRNPA1L2 |
| | IGLON5 |
| | SNX5 |
| | TRIP13 |
| | TRIM71 |
| | TAX1BP3 |
| | KCNQ2 |
| | LHX6 |
| | ERGIC1 |
| | TES |
| | PTDSS2 |
| | LLPH |
| | PCDH8 |
| | C1GALT1 |
| | RADIL |
| | PSMD8 |
| | C3orf26 |
| | TRPC4AP |
| | BCAP31 |
| | TMED3 |
| Olaratumab | UBQLN4 |
| | LRRIQ4 |
| | TAF4 |
| | PCDHB13 |
| | WWC1 |
| | HNRNPL |
| | HPDL |
| | ANKRD2 |
| | EFHD1 |
| | ZNF236 |
| | PTCD1 |
| | MRPS18A |
| | WDR93 |
| | SLC7A6 |
| | GSTM3 |
| | CXorf40B |
| | ERGIC1 |
| | COBL |
| | DHX34 |
| | CHD5 |
| | SP9 |
| | MTPAP |
| | PKP2 |
| | HSD11B2 |
| | SPATA2 |
| Palbociclib | TPT1 |
| | RPL34 |
| | RPS6KA6 |
| | CSNK1G3 |
| | ATP13A4 |
| | ACSS2 |
| | IMMP2L |
| | PMPCB |
| | RDH13 |
| | ATP13A5 |
| | ADI1 |
| | SUCLA2 |
| | MAN1C1 |
| | ARL6 |
| | MAP1LC3A |
| | SLC9A9 |
| | EIF3D |
| | PLS1 |
| | EIF1 |
| | UCP1 |
| | ACSS3 |
| | AHCYL1 |
| | MOBKL2C |
| | RPL4 |
| | RPS20 |
| Pazopanib | SPATA2 |
| | PTCD1 |
| | GSTM3 |
| | DHX34 |
| | PCSK6 |
| | DLL1 |
| | SLC4A2 |
| | CCAR1 |
| | NPBWR1 |
| | SFPQ |
| | ZNF283 |
| | CCDC112 |
| | DLG5 |
| | USP49 |
| | SRRM1 |
| | NFYA |
| | GRID2IP |
| | USP35 |
| | MMP21 |
| | HPDL |
| | OR10H1 |
| | OTOP3 |
| | HDLBP |
| | TDRD12 |
| | ZNRF3 |
| Pexidartinib | MFSD2B |
| | TAF4 |
| | BIRC7 |
| | ALPPL2 |
| | C1orf135 |
| | LRRC42 |
| | HNRNPC |
| | C1GALT1 |
| | BMP8B |
| | MSLN |
| | HIST1H1D |
| | POLQ |
| | SLC35A2 |
| | RGS19 |
| | LHX2 |
| | HNRNPUL1 |
| | PPP1R3G |
| | LRFN4 |
| | WNT10B |
| | GREM1 |
| | CCNA2 |
| | KIF2C |
| | CSTB |
| | RPRD1B |
| | CCNF |
| Pomalidomide | HN1L |
| | CCNF |
| | MNS1 |
| | HNRNPAB |
| | KRT75 |
| | AIMP2 |
| | SFPQ |
| | KRT6B |
| | HPDL |
| | TRIP13 |
| | DPF1 |
| | CHAF1B |
| | KIF2C |
| | DMBX1 |
| | NCAPD2 |
| | HK1 |
| | PPM1G |
| | CCNA2 |
| | SKA1 |
| | PAICS |
| | KIF23 |
| | RADIL |
| | GREM1 |
| | SV2B |
| | SLC7A5 |
| Ponatinib | SND1 |
| | FLNC |
| | CHD5 |
| | SEH1L |
| | CTRB2 |
| | HNRNPA1L2 |
| | IGLON5 |
| | ANKRD40 |
| | KCNK9 |
| | TSPAN15 |
| | SNX5 |
| | TRIP13 |
| | KRT3 |
| | TRIM71 |
| | CCRN4L |
| | PSMG2 |
| | KCNQ2 |
| | ATP5G3 |
| | LHX6 |
| | ERGIC1 |
| | TES |
| | PTDSS2 |
| | PCDH8 |
| | CRCP |
| | PSMD8 |
| Porfimer | FAM83B |
| | KLF14 |
| | KLK8 |
| | CALHM3 |
| | HOXB13 |
| | GMNN |
| | B3GALT5 |
| | NRG3 |
| | PSAPL1 |
| | GSTP1 |
| | SLCO4A1 |
| | CGREF1 |
| | DLX2 |
| | C2orf39 |
| | FAM63B |
| | S100A7 |
| | PSG4 |
| | TNNT3 |
| | KRT6A |
| | TET1 |
| | GPR64 |
| | SPRR3 |
| | MITF |
| | MNT |
| | DIO3 |
| Prexasertib | FBXO40 |
| | CELF4 |
| | PCDHGA7 |
| | ERP44 |
| | GATA5 |
| | NUP214 |
| | HGSNAT |
| | XKR6 |
| | BACH1 |
| | CCBP2 |
| | PCDHA11 |
| | SLC6A3 |
| | ZNF300 |
| | KLK1 |
| | TMEM90A |
| | SLC7A2 |
| | IQGAP1 |
| | ZNF560 |
| | SFTPA1 |
| | ECHDC3 |
| | MITF |
| | ANKDD1A |
| | PCDHGA3 |
| | TLE4 |
| | AGPAT9 |
| Regorafenib | SPATA2 |
| | PTCD1 |
| | GSTM3 |
| | CSTB |
| | PCSK6 |
| | DLL1 |
| | GALNS |
| | SLC4A2 |
| | PIGT |
| | CCAR1 |
| | NPBWR1 |
| | SFPQ |
| | ZNF283 |
| | TNC |
| | PTPN2 |
| | DLG5 |
| | USP49 |
| | SRRM1 |
| | LCTL |
| | SEH1L |
| | NFYA |
| | CTRB2 |
| | GRID2IP |
| | USP35 |
| | MMP21 |
| Romidepsin | SOD2 |
| | TTPAL |
| | VPS26A |
| | AURKC |
| | NID1 |
| | KLF17 |
| | EIF1 |
| | CTH |
| | UBE2D3 |
| | TSC22D2 |
| | RER1 |
| | AGPAT4 |
| | ATP5B |
| | DACT1 |
| | RNF24 |
| | HIST1H2AL |
| | CEBPB |
| | PARVB |
| | PLIN2 |
| | DDX19B |
| | VAMP3 |
| | PGK2 |
| | ACTR2 |
| | GNB1 |
| | RHOC |
| Ruxolitinib | PLXNA3 |
| | CCNB2 |
| | IQGAP3 |
| | PADI3 |
| | C20orf20 |
| | KIF23 |
| | IGF2BP3 |
| | TIPIN |
| | CNPY3 |
| | FOXM1 |
| | KIAA1524 |
| | ZDHHC7 |
| | MFSD2B |
| | HOXB13 |
| | IQGAP3 |
| | TPX2 |
| | SMC4 |
| | XRCC2 |
| | PHLDA2 |
| | NSUN2 |
| | NCAPG2 |
| | POLQ |
| | KDM5C |
| | SLC4A2 |
| | MYBL2 |
| Siltuximab | POMC |
| | ZYG11A |
| | PARP10 |
| | DENND3 |
| | RBM15 |
| | KLK8 |
| | BIRC5 |
| | APOO |
| | INSM2 |
| | MT3 |
| | AMELX |
| | CCR1 |
| | C13orf36 |
| | TRIM48 |
| | PDCD7 |
| | MLL3 |
| | MT1F |
| | LCN15 |
| | HES4 |
| | AIRE |
| | ZC3H18 |
| | TAF3 |
| | OR2L13 |
| | NCR2 |
| | DIAPH2 |
| Sonidegib | ADAMTS14 |
| | TTLL12 |
| | CREB3L1 |
| | TMC4 |
| | NBEAL2 |
| | BMP8B |
| | ERGIC1 |
| | SRPX2 |
| | P4HA3 |
| | AVPR1A |
| | CCND1 |
| | PLEKHF1 |
| | NGF |
| | COL10A1 |
| | LDLRAP1 |
| | PROM2 |
| | TAGLN2 |
| | FOXL1 |
| | TFAP2A |
| | PITRM1 |
| | UNC5A |
| | ANO1 |
| | HTATIP2 |
| | NAGA |
| | SPRY4 |
| Sorafenib | SPATA2 |
| | PTCD1 |
| | GSTM3 |
| | DHX34 |
| | CSTB |
| | PCSK6 |
| | FLNC |
| | DLL1 |
| | GALNS |
| | SLC4A2 |
| | PIGT |
| | CCAR1 |
| | NPBWR1 |
| | SFPQ |
| | ZNF283 |
| | TNC |
| | CCDC112 |
| | PTPN2 |
| | DLG5 |
| | USP49 |
| | SRRM1 |
| | LCTL |
| | SEH1L |
| | NFYA |
| | CTRB2 |
| Sunitinib | SPATA2 |
| | PTCD1 |
| | GSTM3 |
| | DHX34 |
| | PCSK6 |
| | FLNC |
| | DLL1 |
| | MFSD2B |
| | SLC4A2 |
| | PIGT |
| | CCAR1 |
| | NPBWR1 |
| | SFPQ |
| | ZNF283 |
| | CCDC112 |
| | DLG5 |
| | USP49 |
| | SRRM1 |
| | NFYA |
| | GRID2IP |
| | USP35 |
| | TAF4 |
| | MMP21 |
| | HPDL |
| | OR10H1 |
| Vandetanib | LAMB4 |
| | NUCB1 |
| | CABP4 |
| | SERTAD1 |
| | BLCAP |
| | EIF2B3 |
| | ZYX |
| | PCDH8 |
| | KCNJ14 |
| | AVEN |
| | TNC |
| | NRBF2 |
| | TOMM40L |
| | RFPL4B |
| | RADIL |
| | FBXL13 |
| | DNAJC8 |
| | ZNF701 |
| | MYCBP |
| | ZNF581 |
| | ADAM30 |
| | LRRC39 |
| | SUSD1 |
| | CRISPLD2 |
| | ATP5SL |
| Vorinostat | SOD2 |
| | TTPAL |
| | VPS26A |
| | AURKC |
| | NID1 |
| | KLF17 |
| | EIF1 |
| | CTH |
| | TSC22D2 |
| | RER1 |
| | AGPAT4 |
| | ATP5B |
| | DACT1 |
| | RER1 |
| | RNF24 |
| | HIST1H2AL |
| | CEBPB |
| | PARVB |
| | DDX19B |
| | VAMP3 |
| | GNB1 |
| | PGK2 |
| | ADH1B |
| | ACTR2 |
| | GNB1 |
| Exemestane | MMP21 |
| | ZNF662 |
| | ANKRD1 |
| | ALPP |
| | H2AFY2 |
| | FGF10 |
| | DCAF12L1 |
| | KIAA1549 |
| | RBM12 |
| | PTPRN2 |
| | LRRC8A |
| | TSPAN14 |
| | MYH11 |
| | SLC4A7 |
| | HECW2 |
| | NKD1 |
| | ARHGDIG |
| | RC3H2 |
| | MLL3 |
| | DRD1 |
| | TAF1L |
| | SLC7A6 |
| | ZXDB |
| | VSIG2 |
| | TMEFF2 |
| Letrozole | MMP21 |
| | ZNF662 |
| | ANKRD1 |
| | ALPP |
| | H2AFY2 |
| | FGF10 |
| | DCAF12L1 |
| | KIAA1549 |
| | RBM12 |
| | PTPRN2 |
| | LRRC8A |
| | TSPAN14 |
| | MYH11 |
| | SLC4A7 |
| | HECW2 |
| | NKD1 |
| | ARHGDIG |
| | RC3H2 |
| | MLL3 |
| | DRD1 |
| | TAF1L |
| | SLC7A6 |
| | ZXDB |
| | VSIG2 |
| | TMEFF2 |
| Decitabine | PCDHGA7 |
| | MLLT4 |
| | CHD5 |
| | WFIKKN2 |
| | DRD1 |
| | BACH1 |
| | TUB |
| | PDZD4 |
| | CLDN18 |
| | PCDHGA10 |
| | PCDHGA6 |
| | BAZ2A |
| | KIAA0355 |
| | ESYT2 |
| | PRSS33 |
| | ZNF300 |
| | CAPS2 |
| | RHOBTB3 |
| | PCDHGA5 |
| | KDM6B |
| | PRDM2 |
| | ZNF573 |
| | GPR114 |
| | AADAC |
| | ANKRD17 |
| Panobinostat | SOD2 |
| | TTPAL |
| | VPS26A |
| | AURKC |
| | KLF17 |
| | CYTH3 |
| | EIF1 |
| | CTH |
| | TSC22D2 |
| | RER1 |
| | AGPAT4 |
| | ATP5B |
| | DACT1 |
| | RNF24 |
| | HIST1H2AL |
| | CEBPB |
| | MFI2 |
| | DDX19B |
| | VAMP3 |
| | GNB1 |
| | PGK2 |
| | GFRA2 |
| | NR5A1 |
| | RNF24 |
| | MAN2A1 |
| Osimertinib | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Erlotinib | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Gefitinib | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Necitumumab | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Panitumumab | TOMM40L |
| | NTRK1 |
| | ALS2 |
| | AMMECR1L |
| | B4GALT5 |
| | PACS1 |
| | CSMD3 |
| | KLHL34 |
| | AIRE |
| | VPS13B |
| | LCT |
| | CHRFAM7A |
| | PGCP |
| | MTBP |
| | RUFY1 |
| | RIMS2 |
| | TRAF3 |
| | TRIM71 |
| | TMEM74 |
| | MATN3 |
| | C14orf145 |
| | NIPAL2 |
| | LARP1 |
| | LSM11 |
| | CLIP2 |
| Trametinib | CPT2 |
| | RPL5 |
| | CTDSPL2 |
| | MAP1LC3A |
| | HBS1L |
| | USP3 |
| | MRTO4 |
| | ELOVL6 |
| | PRKACG |
| | TADA2A |
| | CUL5 |
| | ME3 |
| | STXBP3 |
| | DHX35 |
| | CSNK1G3 |
| | RBBP4 |
| | MRE11A |
| | NAA16 |
| | BCAT2 |
| | RAB33B |
| | DDX46 |
| | CABP5 |
| | WDR26 |
| | LACTB2 |
| | PRPSAP1 |
| Temsirolimus | PRKCG |
| | DNAJB12 |
| | CDK16 |
| | C20orf118 |
| | STK4 |
| | CTH |
| | NOX1 |
| | DNAJC2 |
| | FGR |
| | TAF12 |
| | DDOST |
| | RPS6KA1 |
| | TSSK3 |
| | RHEBL1 |
| | SLC7A6 |
| | MYLK4 |
| | KMO |
| | PSMB9 |
| | HERC4 |
| | SGPL1 |
| | PTPN6 |
| | SLC2A5 |
| | NOL10 |
| | TRNAU1AP |
| | VAC14 |
| Ramucirumab | PIGT |
| | KNTC1 |
| | SNX5 |
| | LEP |
| | TAX1BP3 |
| | FAM83D |
| | ERGIC1 |
| | FGF5 |
| | LRRC42 |
| | MYC |
| | KCNA7 |
| | HOXC8 |
| | CDC25B |
| | P4HA3 |
| | KIF3C |
| | GREM1 |
| | CDC25B |
| | CDC25B |
| | LAMB1 |
| | FLNC |
| | FHOD1 |
| | SLC7A6 |
| | ZFHX4 |
| | ITGA5 |
| | NUMBL |
| Ribociclib | TPT1 |
| | RPL34 |
| | RPS6KA6 |
| | CSNK1G3 |
| | ATP13A4 |
| | ACSS2 |
| | IMMP2L |
| | PMPCB |
| | RDH13 |
| | ATP13A5 |
| | ADI1 |
| | SUCLA2 |
| | MAN1C1 |
| | ARL6 |
| | MAP1LC3A |
| | SLC9A9 |
| | EIF3D |
| | PLS1 |
| | EIF1 |
| | UCP1 |
| | ACSS3 |
| | AHCYL1 |
| | MOBKL2C |
| | RPL4 |
| | RPS20 |

Using the above operations and methods using SL-scores, results from several data analyses have been conducted and provided herein. For example, FIGS. 1E-1J illustrate results of four melanoma cohorts treated with BRAF inhibitors identified through the operations above of FIGS. 1A-1B. Applying SELECT, the 25 most significant SL partners of BRAF are identified, where the number 25 was determined from training on one single dataset and kept fixed thereafter in all targeted therapies predictions. As expected, responders have higher SL-scores than non-responders in the three melanoma-BRAF cohorts for which therapy response data is available, as shown in the graph of FIG. 1E, where SL-scores are significantly higher in responders (green) vs non-responders (red), based on Wilcoxon ranksum test after multiple hypothesis correction. Quantifying the predictive power via the use of the standard area under the receiver operating characteristics curve (Area Under the Curve (AUC) of the Receiver Operating Characteristic (ROC) curve) measure, AUCs greater than 0.7 occur in all three datasets, and an aggregate performance of AUC=0.71 when the three cohorts are merged as shown in the graph of FIG. 1F. As some datasets do not have a balanced number of responders and non-responders, the resulting performance may be additionally quantified via precision-recall curves (often used as supplement to the routinely used ROC curves). As evident from the latter, one can choose a single classification threshold that successfully captures most true responders while misclassifying less than half of the non-responders. Even though all patients in these three cohorts have the same mutation, there is still a large variability in their response, which is captured by the method to predict the patient who will respond better to BRAF inhibition.

SL based prediction accuracy levels are overall higher compared to those obtained by several published transcriptomic based predictors, including the proliferation index, IFNg signature, cytolytic score, or the expression of the drug target gene itself (BRAF in this case). FIG. 1G includes bar graphs illustrating the predictive accuracy in terms of AUC of ROC curve (Y-axis) of SL-based predictors (red) and controls including several known transcriptomics-deduced metrics (IFNg signature, proliferation index, cytolytic score, and the drug target expression levels) and several interaction-based “SL-like” scores (based on randomly chosen partners, randomly chosen PPI partners of the drug target gene(s), the identified SL partners of other cancer drugs, and experimentally identified SL partners) in the three BRAF inhibitor cohorts (X-axis). As shown in the graph, SL based prediction accuracy levels are better than other interaction-based scores, including the fraction of down-regulated randomly selected genes, the fraction of in vitro experimentally determined SL partners, the fraction of the identified SL partners of other drugs, or the fraction of down-regulated protein-protein interaction partners (all of sizes similar to the SL set; empirical P<0.001). The patients with high SL-score (defined as those in the top tertile) show significantly higher rate of response than the overall response rate, and the patients with low SL-score (in the bottom tertile) show the opposite trend, as illustrated in the bar graphs of FIG. 1H. More particularly, the bar graphs of FIG. 1H show the fraction of responders in the patients with high SL-scores (top tertile; green) and low SL-scores (bottom tertile; purple). The grey line denotes the response rate of each cohort, and the stars denote the hypergeometric significance of enrichment of responders in the high-SL group and depletion of responder in the low-SL group (compared to their baseline frequency in the cohort).

It is noted that patients with higher SL-scores showed significantly better treatment outcome in terms of progression-free survival in one of the datasets analyzed above where this data was available to us, as shown in the Kaplan-Meier curves of FIG. 1I, in which the survival of patients with low (yellow) vs high (blue) BRAF SL-scores (top vs. bottom tertile SL-score) GSE50509. Moreover, integrated analysis of large-scale BRAF inhibitor clinical trials shows that SL-score is associated with significantly improved progression-free survival, as shown in the Kaplan-Meier curves of FIG. 1J, in which patients with high SL-scores show better prognosis, as expected. The logrank P-value and median survival difference are denoted in the graph. As expected, the SL partners of BRAF are found to be enriched with the functional annotation ‘regulation of GTPase mediated signal transduction’.

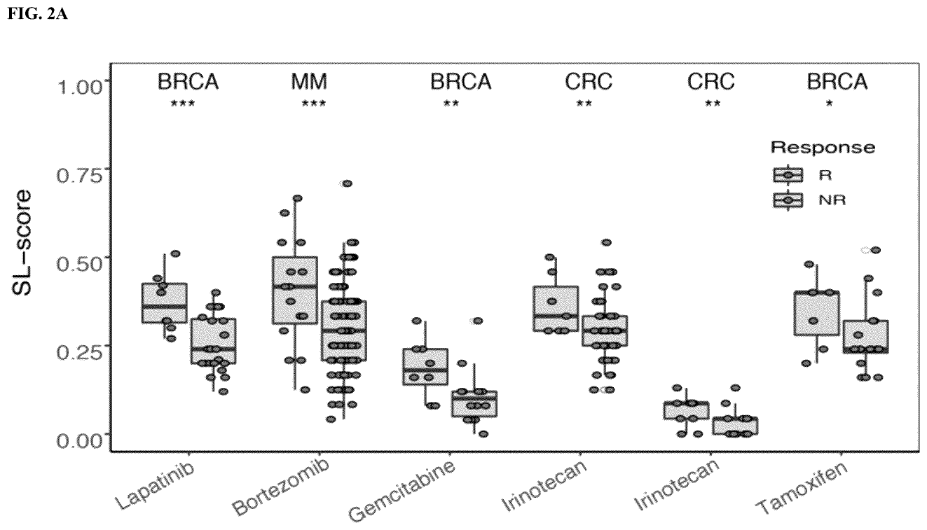

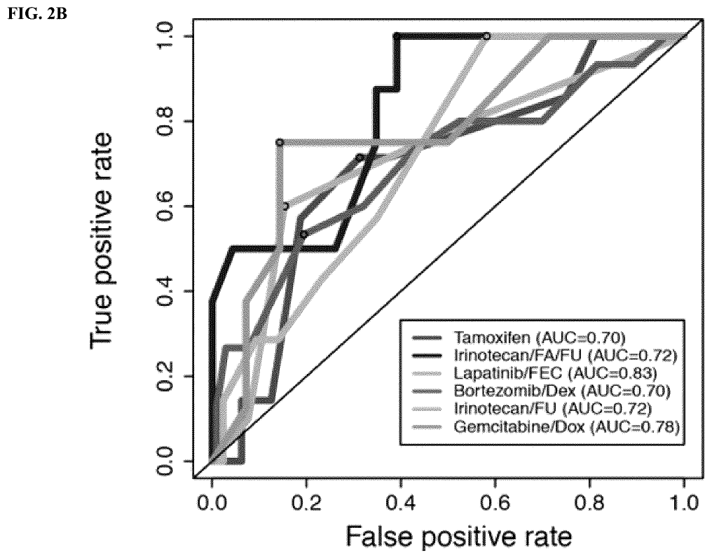

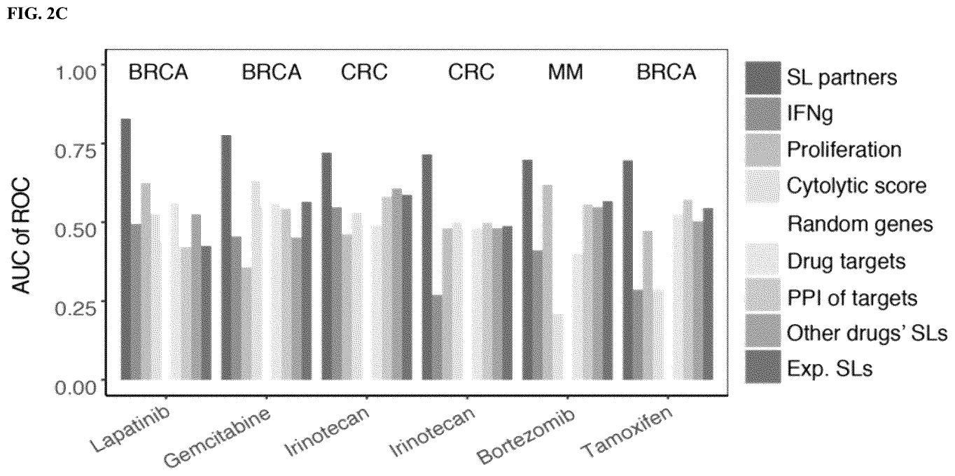

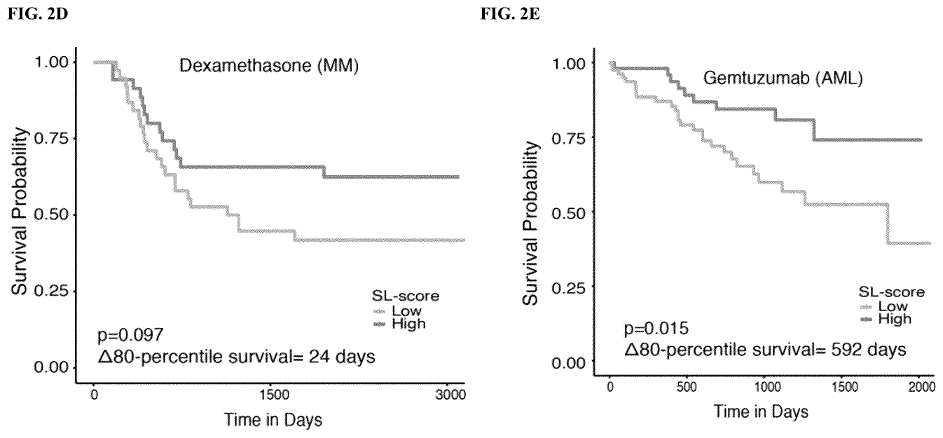

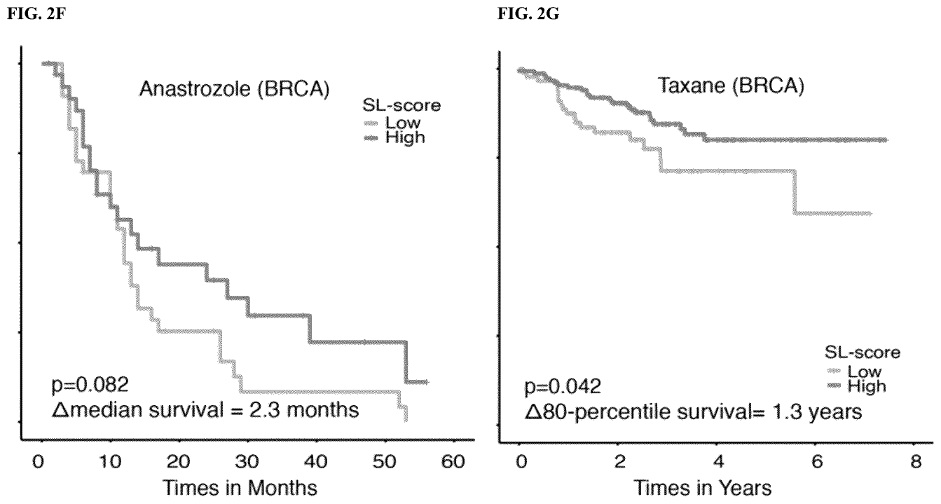

FIGS. 2A-2H illustrate prediction accuracy results identified through the SL-based method of FIGS. 1A-1B on chemo and targeted therapy in different cancer types. In particular, a collection of publicly available datasets from clinical trials of cytotoxic agents and targeted cancer therapies, each one containing both pre-treatment transcriptomics data and therapy response information, may be accessed. This compendium of data includes breast cancer patients treated with lapatinib, tamoxifen, and gemcitabine; colorectal cancer patients treated with irinotecan, multiple myeloma patients treated with bortezomib acute myeloid leukemia treated with gemtuzumab, and a multiple myeloma cohort treated with dexamethasone. To determine the accuracy of the SL-based method, the SL interaction partners of the drug targets in the datasets may be identified and an SL-score in each sample using the SL partners of the corresponding drugs may be computed. It is noted that the framework mostly fails in predicting the response to cytotoxic agents, obtaining AUC>0.7 in only 3 out of 11 of these datasets (where information is available). This is not surprising given that prediction accuracy may depend on the specificity and correct identification of the drug targets, and cytotoxic agents typically have a multitude of targets, often ill-defined, a major difference from the more recently developed targeted and checkpoint therapies. Indeed, higher SL-scores may be associated with better response in 3 out of 5 of targeted therapy datasets. As illustrated in the graph of FIG. 2A, the result for the therapies is successfully predicted (AUC's all greater than 0.7). More particularly, the graph of FIG. 2A illustrates that SL-scores are significantly higher in responders (green) vs non-responders (red), based on Wilcoxon ranksum test after multiple hypothesis correction. For false discovery rates: * denotes 10%, ** denotes 5%, *** denotes 1%, and **** denotes 0.1% in the graph. Also, cancer types are noted on the top of each dataset.

The graph of FIG. 2B illustrates ROC curves for breast cancer patients treated with lapatinib (GSE66399), tamoxifen (GSE16391), gemcitabine (GSE8465), colorectal cancer patients treated with irinotecan (GSE72970, GSE3964), and multiple myeloma patients treated with bortezomib (GSE68871). The circles of the graph of FIG. 2B denote the point of maximal F1-score. Further, the bar graphs of FIG. 2C show the predictive accuracy in terms of AUCs (Y-axis) of SL-based predictors and a variety of controls specified above in relation to FIG. 1E (X-axis). As shown in FIGS. 2B and 2C, the predictive performance of a variety of expression-based control predictors is random. As shown, patients with high SL-scores (within top tertile) have significantly higher response rates than the overall response rates, and the patients with low SL-scores (within bottom tertile) show the opposite trend. FIGS. 2D-2G illustrate Kaplan-Meier curves depicting the survival of patients with low vs high SL-scores of small cell lung cancer patients treated with dexamethasone (FIG. 2D), acute myeloid leukemia patients treated with gemtuzumab (FIG. 2E), breast cancer treated with anastrozole (GSE41994) (FIG. 2F), and breast cancer cohorts treated with taxane-anthracycline GSE25055 (FIG. 2G), where X-axis denotes survival time and Y-axis denotes the probability of survival. Patients with high SL-scores (top-tertile, blue) show better prognosis than the patients with low SL-scores (bottom tertile, yellow), as expected. The logrank P-values and median survival differences (or 80-percentile survival differences if survival exceeds 50% at the longest time point) are denoted in the figure. Tumor type abbreviations: MM, multiple myeloma; CRC, colorectal cancer; BRCA, breast invasive carcinoma; AML, acute myeloid leukemia.

In addition to the SL approach, the above operations may also be used for SR-based prediction of response to a therapy or drug. For example, the ability of the SELECT framework to predict clinical response to checkpoint inhibitors is conducted and discussed herein. In particular, to identify the SR interaction partners that are predictive of the response to anti-PD1/PDL1 and anti-CTLA4 therapy, the published pipelines may be modified to take into account the characteristics of immune checkpoint therapy. For anti-PD1/PDL1 therapy, where the antibody blocks the physical interaction between PD1 and PDL1, consideration of the interaction term (i.e. the product of PD1 and PDL1 gene expression values) to identify the SR partners of the treatment may be used. For anti-CTLA4 therapy, where the precise mechanism of action involves several ligand/receptor interactions, focus may be on the CTLA4 itself, using its protein expression levels as they are likely to better reflect the activity than the mRNA levels Using this immune-tailored version of the framework, analysis of the TCGA data to identify the SL and SR partners of PD1/PDL1 and of CTLA4 is performed. In general, SR interactions denote such genetic interactions where inactivation of the target gene is compensated by downregulation (or upregulation) of the partner rescuer gene. Given a drug and tumor transcriptomics data from an individual patient, the fraction f of SR partners that are downregulated (or upregulated) may be quantified. Definition of 1−f as the SR-score may be assumed, where tumors with higher SR scores have less “active” rescuers are hence expected to respond better to the given checkpoint therapy.

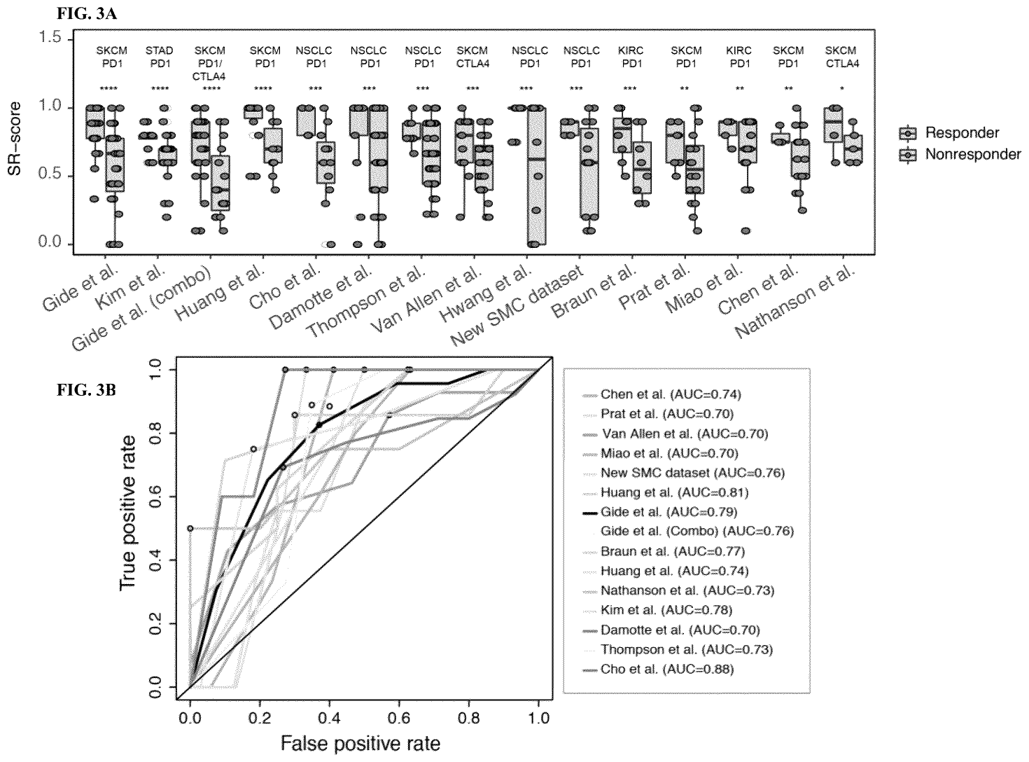

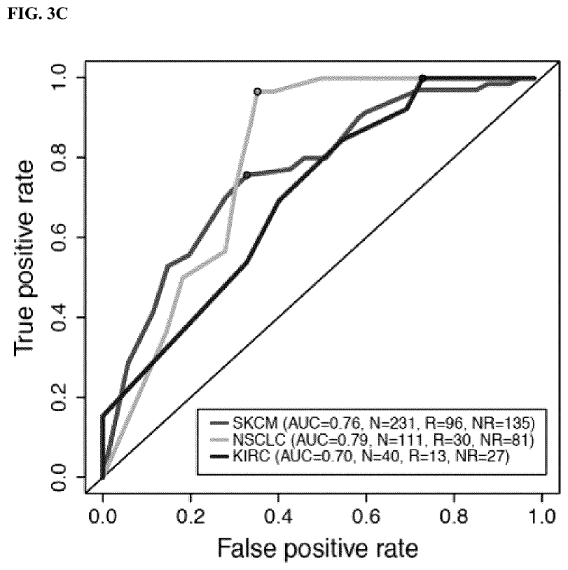

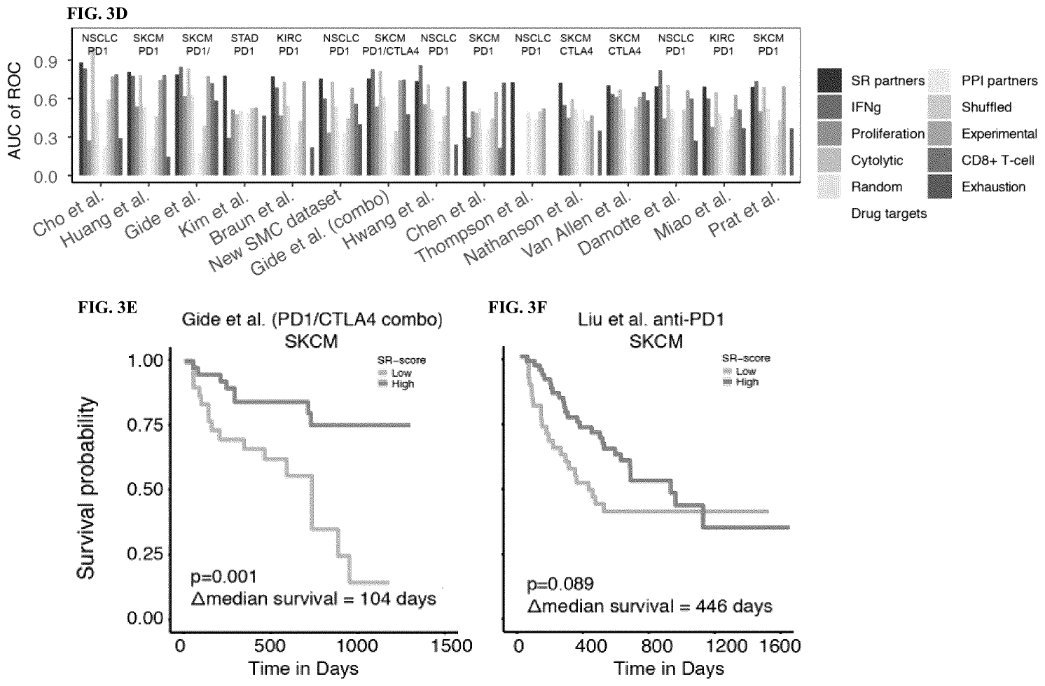

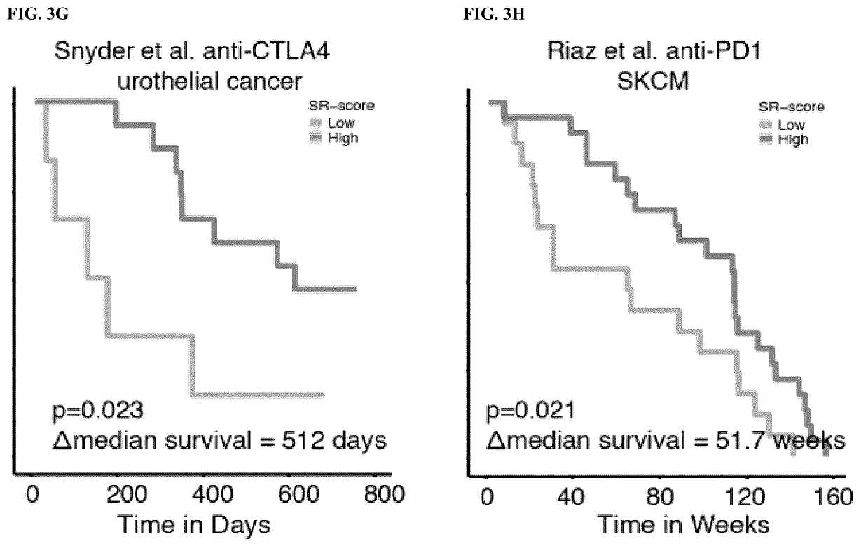

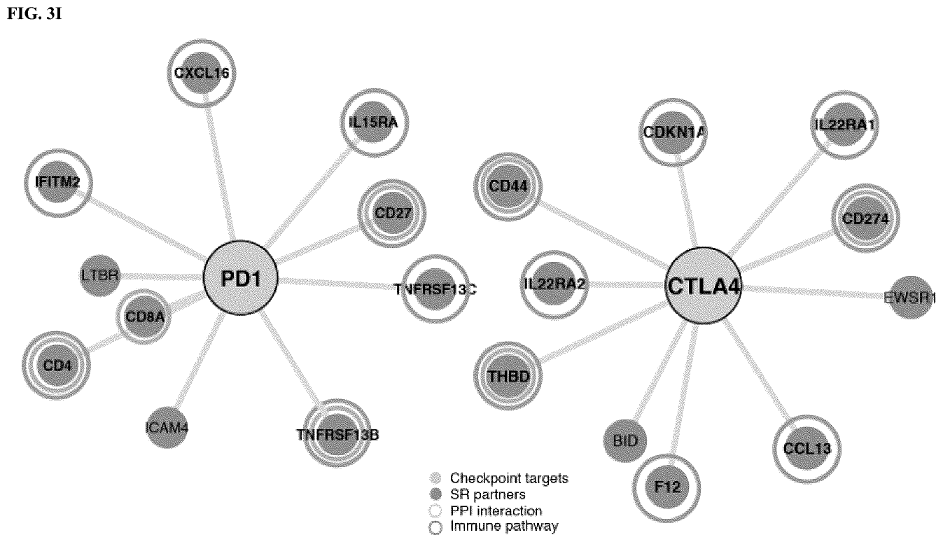

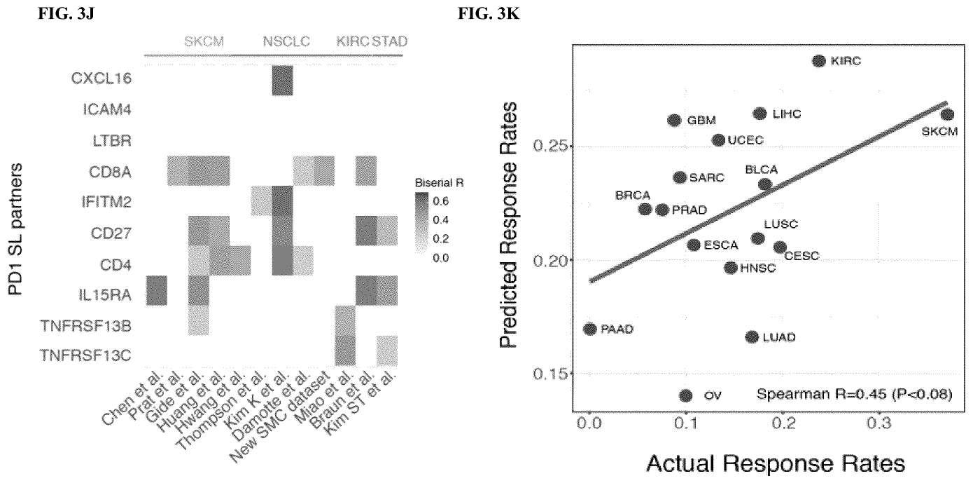

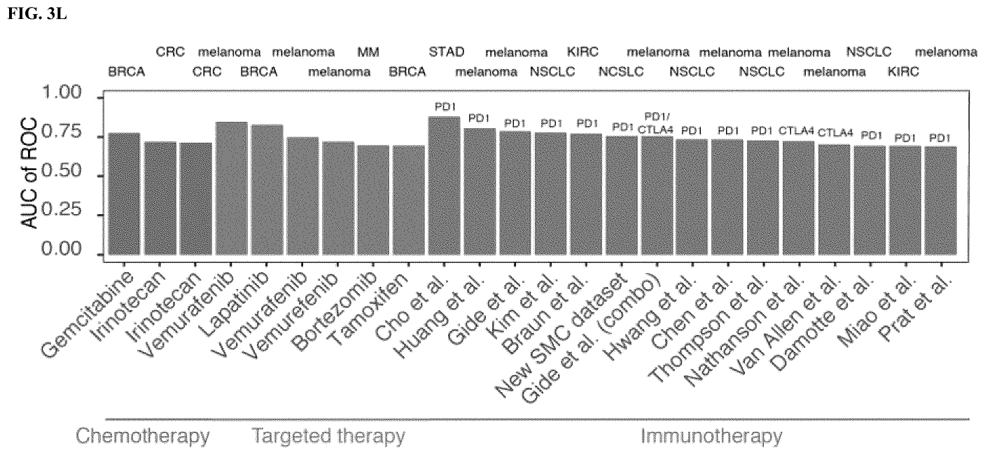

To evaluate the accuracy of SR-based predictions, a collected set of 21 immune checkpoint therapy datasets, comprising 1050 patients, may be gathered that includes both pre-treatment transcriptomics data and therapy response information (either by RECIST or Progression-Free Survival (PFS)). Tumor types represented in these datasets include melanoma, non-small cell lung cancer, renal cell carcinoma, metastatic gastric cancer, and urothelial carcinoma cohorts treated with anti-PD1/PDL1 or anti-CTLA4-, or their combination. FIGS. 3A-3M illustrate prediction accuracy results identified through the SR-based method of FIGS. 1A-1B on an array of different therapies and cancer types. In particular, FIG. 3A is a graph illustrating SR-scores significantly higher in responders (green) vs non-responders (red) based on Wilcoxon ranksum test after multiple hypothesis correction. For false discovery rates: * denotes 20%, ** denotes 10%, *** denotes 5%, and **** denotes 1%. Cancer types are noted on the top of each dataset. Results are shown for melanoma (found in Analysis of Immune Signatures in Longitudinal Tumor Samples Yields Insight into Biomarkers of Response and Mechanisms of Resistance to Immune Checkpoint Blockade, Cancer Discov 6, 827-837 to Chen, P. L., Roh, W., Reuben, A., Cooper, Z. A., Spencer, C. N., Prieto, P. A., Miller, J. P., Bassett, R. L., Gopalakrishnan, V., Wani, K., et al. (2016); Primary, and Acquired Resistance to Immune Checkpoint Inhibitors in Metastatic Melanoma, Clin Cancer Res 24, 1260-1270 to Gide, T. N., Wilmott, J. S., Scolyer, R. A., and Long, G. V. (2018); Integrative molecular and clinical modeling of clinical outcomes to PD1 blockade in patients with metastatic melanoma, Nat Med 25, 1916-1927 to Liu, D., Schilling, B., Liu, D., Sucker, A., Livingstone, E., Jerby-Amon, L., Zimmer, L., Gutzmer, R., Satzger, I., Loquai, C., et al. (2019); Somatic Mutations and Neoepitope Homology in Melanomas Treated with CTLA-4 Blockade, Cancer Immunol Res 5, 84-91 to Nathanson, T., Ahuja, A., Rubinsteyn, A., Aksoy, B. A., Hellmann, M. D., Miao, D., Van Allen, E., Merghoub, T., Wolchok, J. D., Snyder, A., et al. (2017); and Immune-Related Gene Expression Profiling After PD-1 Blockade in Non-Small Cell Lung Carcinoma, Head and Neck Squamous Cell Carcinoma, and Melanoma, Cancer Res 77, 3540-3550 to Prat, A., Navarro, A., Pare, L., Reguart, N., Galvan, P., Pascual, T., Martinez, A., Nuciforo, P., Comerma, L., Alos, L., et al. (2017)), non-small cell lung cancer (found in Immune gene signatures for predicting durable clinical benefit of anti-PD-1 immunotherapy in patients with non-small cell lung cancer, Sci Rep 10, 643 to Hwang, S., Kwon, A. Y., Jeong, J. Y., Kim, S., Kang, H., Park, J., Kim, J. H., Han, O. J., Lim, S. M., and An, H. J. (2020)), renal cell carcinoma (Genomic correlates of response to immune checkpoint therapies in clear cell renal cell carcinoma, Science 359, 801-806 to Miao, D., Margolis, C. A., Gao, W., Voss, M. H., Li, W., Martini, D. J., Norton, C., Bosse, D., Wankowicz, S. M., Cullen, D., et al. (2018)), and metastatic gastric cancer (Comprehensive molecular characterization of clinical responses to PD-1 inhibition in metastatic gastric cancer, Nat Med 24, 1449-1458 to Kim, S. T., Cristescu, R., Bass, A. J., Kim, K. M., Odegaard, J. I., Kim, K., Liu, X. Q., Sher, X., Jung, H., Lee, M., et al. (2018)) treated with anti-PD1/PDL1, anti-CTLA4 and their combination, and our new lung adenocarcinoma cohort treated with anti-PD1.

FIG. 3B is a graph illustrating ROC curves showing the prediction accuracy obtained with the SR-scores across the 15 different datasets, with stars denoting the point of maximal F1-score. As shown, higher SR-scores are associated with better response to immune checkpoint blockade with AUCs greater than 0.7 in 15 out of 18 datasets, where RECIST information is available and their type-specific aggregation for melanoma, non-small cell lung cancer and kidney cancer in FIG. 3C. FIG. 3D illustrates a bar graph showing the predictive accuracy in terms of AUC (Y-axis) of SR-based predictors and controls across different cohorts (X-axis). Notably, the framework remains predictive when multiple datasets of the same cancer types are combined for melanoma, non-small cell lung cancer, and kidney cancer. As shown, the prediction accuracy of SR-scores is overall superior to a variety of expression-based controls, including T-cell exhaustion markers and the estimated CD8+ T-cell abundance. As expected, the patients with high SR-scores (in the top tertile) are enriched with responders, while the patients with low SR-scores (in the bottom tertile) are enriched with non-responders. The SR-scores are also predictive of either progression-free or overall patient survival in the datasets analyzed above (anti-PD1/anti-CTLA4 combination (shown in the Kaplan-Meier curve depicting the survival of patients with low vs high SR-scores in anti-PD1/CTLA4 combination-treated melanoma of FIG. 3E), nivolumab/pembrolizumab-treated melanoma cohorts (shown in the curve of FIG. 3F), atezolizumab-treated urothelial cancer (shown in the curve of FIG. 3G), nivolumab-treated melanoma cohorts (shown in the curve of FIG. 3H). In each curve, patients with high SR-scores (blue; over top tertile) show better prognosis than the patients with low SR-scores (yellow; below bottom tertile), and the logrank P-values and median survival differences (or 80-percentile survival differences if survival exceeds 50% at the longest time point) are denoted.

FIG. 3I illustrates the SR partners of PD1 (left) and CTLA4 (right), where red circles denote SR partners, yellow circles denote checkpoint targets, purple circles denote genes that belong to immune pathways, and cyan circles denote a protein physical interaction with PD1 or CTLA4, respectively. The predicted SR partners of PD1 and CTLA4 may enriched for T-cell apoptosis and response to IL15, including key immune genes such as CD4, CD8A, and CD274, and PPI interaction partners of PD1 and CTLA4 such as CD44, CD27 and TNFRSF13B. The heatmap of FIG. 3J shows the association of individual SR partners' gene expression (Y-axis) with anti-PD1 response in the 12 clinical trial cohorts (X-axis). The significant point-biserial correlation coefficients are color-coded (P<0.1), and the cancer types of each cohort are denoted on the top of the heatmap. As shown, the contribution of individual SR partners to the response prediction is different across different datasets from different cancer types, where CD4, CD27, and CD8A play an important role in many samples. Taken together, these results testify that the SR partners of PD1 and CTLA4 serve as effective biomarkers for checkpoint response across a wide range of cancer types.

The graph of FIG. 3K illustrates the objective response rates among TCGA patients predicted by the SR-scores (Y-axis) correlated with the actual objective response rates of independent datasets of similar cancer types observed in the pertaining clinical trials (X-axis), with a regression line (blue). The above results, taken together, show that, adding to the existing determinants of response and resistance to checkpoint therapy in melanoma, SR-scores are robust predictors of response to checkpoint therapy across many different cancer types.

To study if tumor-specific SR scores can explain the variability observed in the objective response rates (ORR) of different tumor types to immune checkpoint therapy, the SR-scores for anti-PD1 therapy for each tumor sample in the TCGA may be computed. Based on the latter and the threshold for determining responders, the fraction of predicted responders in each cancer type in the TCGA cohort may be computed. A comparison of these predicted fractions to the actual ORR may be collected from anti-PD1 clinical trials of 16 cancer types. Notably, these two measures significantly correlate, demonstrating that SR-scores are effective predictors of ORR to checkpoint therapy in aggregate across different cancer types.

Summed up the three classes of the drugs studied, the genetic interaction-based approach achieves an AUC greater than 0.7 predictive performance levels in 24 out of 35 datasets containing RECIST response information, spanning 3 out of 12 non-targeted cytotoxic agents, 6 out of 8 targeted therapies and 15 out of 18 immunotherapy cohorts (including our new SMC dataset). More particularly, FIG. 3M includes bar graphs showing the overall predictive accuracy of genetic interaction-based predictors (for which we could determine the AUCs given RECIST response data, Y-axis) for chemotherapy (red), targeted therapy (green) and immunotherapy (purple) in 23 different cohorts encompassing 7 different cancer types and 12 treatment options (X-axis). Tumor type abbreviations include: UCEC, uterine corpus endometrial carcinoma; STAD, stomach adenocarcinoma, SKCM, skin cutaneous melanoma; SARC, sarcoma; PRAD, prostate adenocarcinoma; PAAD, pancreatic adenocarcinoma; OV, ovarian serous cystadenocarcinoma; NSCLC, non-small cell lung cancer; LUAD, lung adenocarcinoma; LUSC, lung squamous cell carcinoma; LIHC, liver hepatocellular carcinoma; KIRC, kidney renal clear cell carcinoma; HNSC, head-neck squamous cell carcinoma; GBM, glioblastoma multiforme; ESCA, esophageal carcinoma; CESC, cervical squamous cell carcinoma and endocervical adenocarcinoma; BRCA, breast invasive carcinoma; and BLCA, bladder carcinoma. Adding the 8 additional datasets where SL/SR-score is predictive of progression-free or overall survival (1 chemo-, 4 targeted- and 3 immuno-therapy), the SELECT framework is predictive in 32 out of 45 cohorts (>70%), and 28 out of 33 (>80%) among the targeted and checkpoint therapies. Notably, these accuracies are markedly better than those obtained using a range of control predictors.

Still another evaluation of the SELECT approach may be conducted utilizing a multi-arm basket clinical trial setting that incorporates transcriptomics data for cancer therapy in adult patients with advanced solid tumors. This multi-center study may include an arm recommending treatment based on actionable mutations in a panel of cancer driver genes and another based on the patients' transcriptomics data. In the evaluation performed, consideration of gene expression data of 71 patients with 50 different targeted treatments (single or combinations) for which significant SL partners were identified may be processed. Of the patient data, one patient had a complete response, 7 had a partial response and 11 were reported to have stable disease (labeled as responders), while 52 had progressive disease (labeled as non-responders).

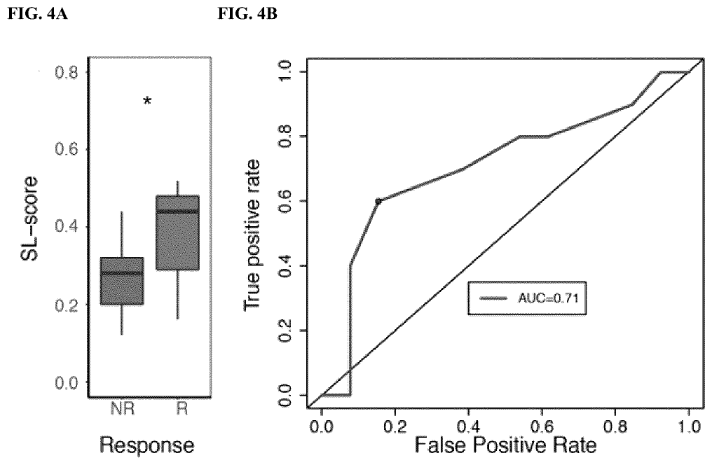

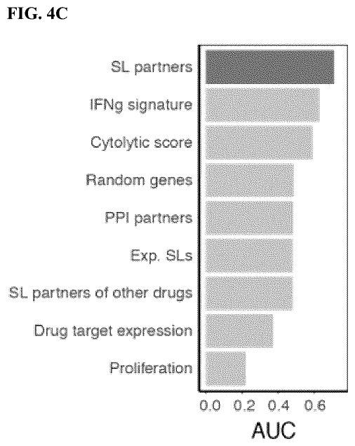

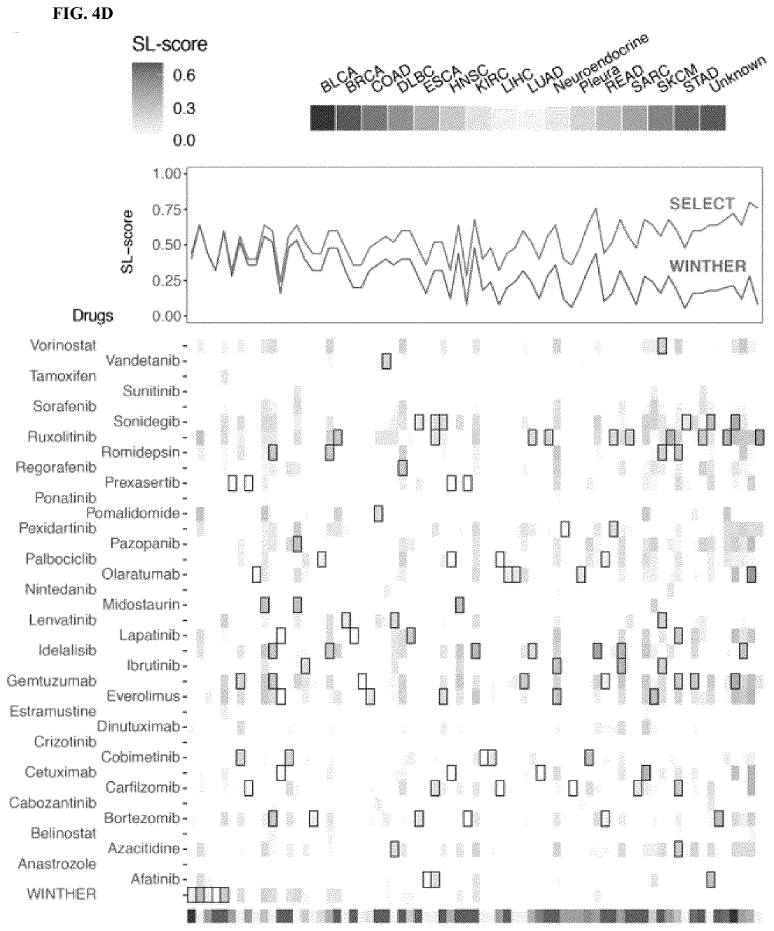

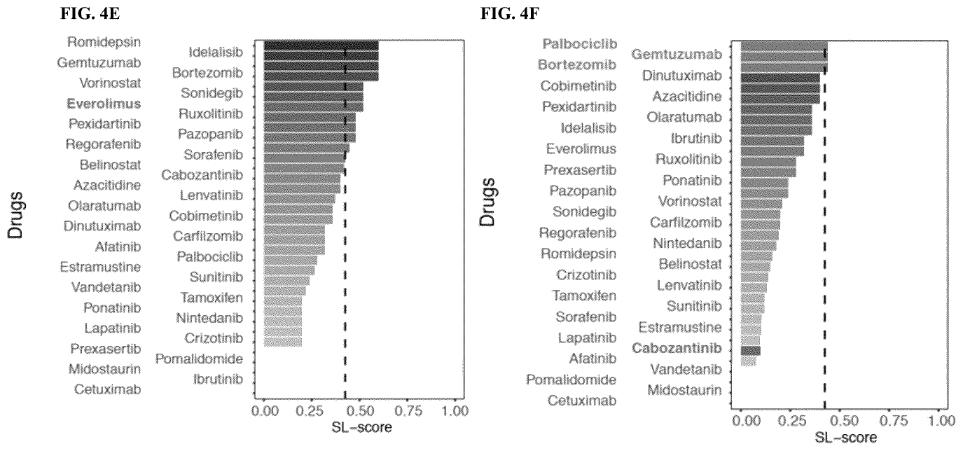

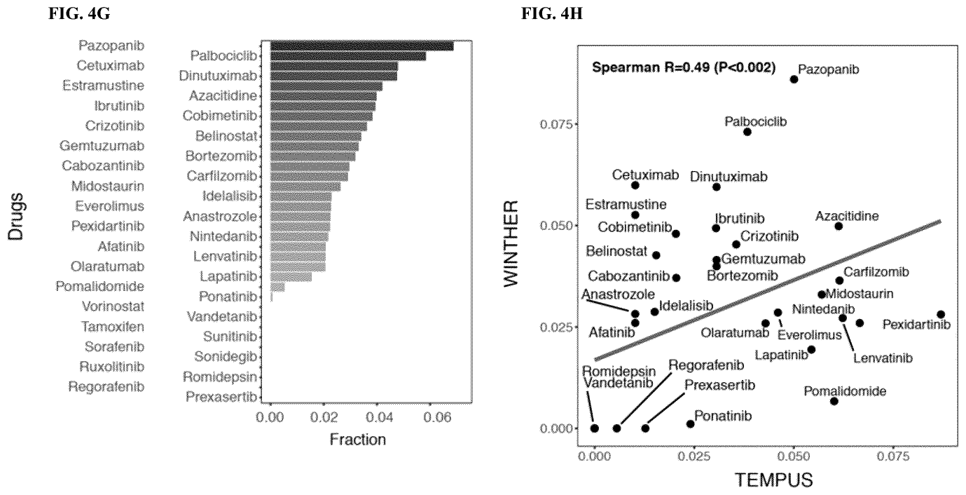

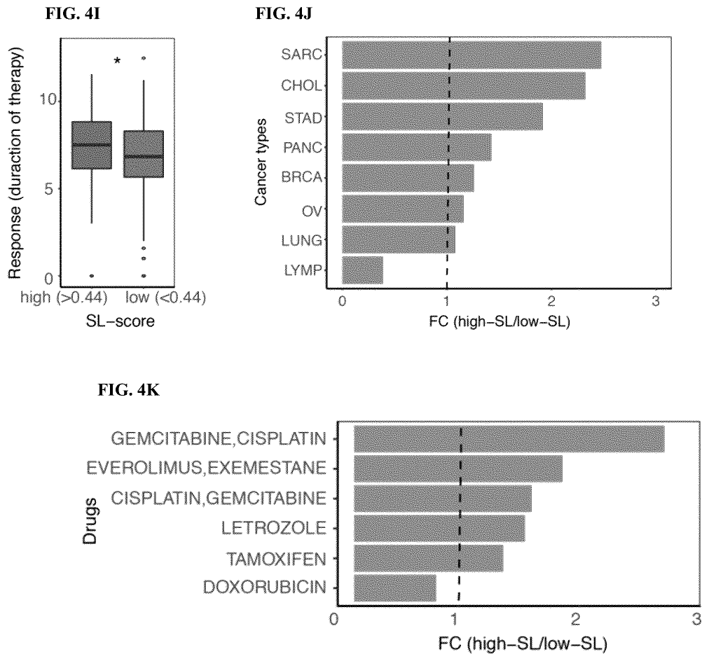

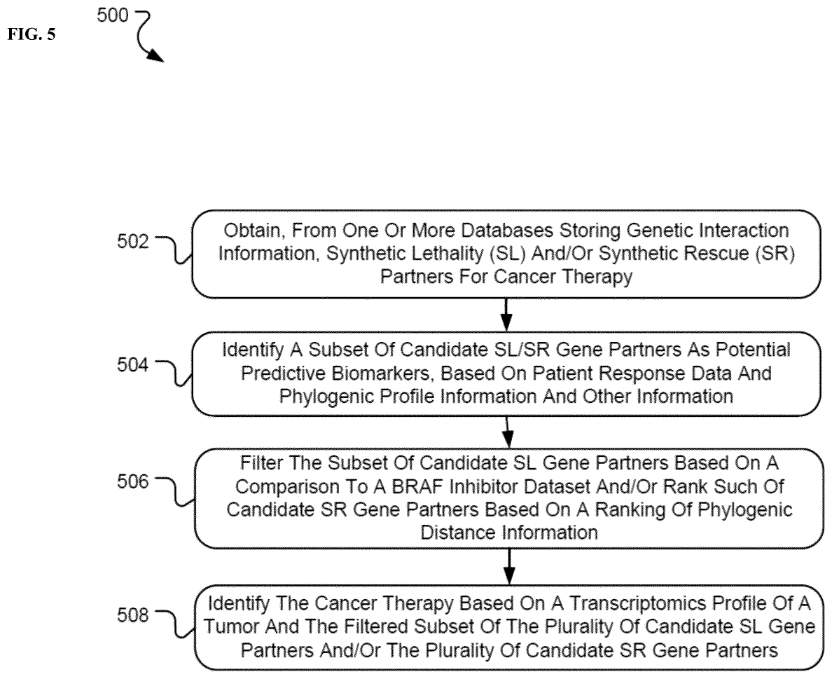

Applying the SELECT approach discussed above, SL partners for each of the drugs prescribed in the study may be first identified. Confirmation that the resulting SL-scores of the therapies used in the trial as significantly higher in responders than non-responders is illustrated in the graph of FIG. 4A, with responders (CR, PR, and SD; red) show significantly higher SL-scores compared to non-responders (PD; green). Notably, the SL-scores of the drugs given to each patient are predictive of the actual responses observed in the trial (AUC=0.71) as illustrated in the graph of FIG. 4B. More particularly, the ROC plot of FIG. 4B shows that the SL-scores are predictive of response to the different treatments prescribed at the trial (AUC of ROC=0.71). The black dot denotes the point of maximal F1-score (SL-score=0.44). With an SL-score of chosen as the optimal threshold with maximal F1-score. The bar graphs of FIG. 4C further show the predictive accuracy in terms of AUC (X-axis) of SL-based predictors and different controls (Y-axis). As shown in the figure, the prediction accuracy of SL-score is superior to that of control expression-based predictors.

FIG. 4D illustrates a comparison of the SL-scores (Y-axis) of the treatments actually prescribed in the examined trial (blue) and the SL-scores of the best therapy identified by our approach (red) across all 71 patients, and the samples are presented in the order of the differences in the two SL-scores. A more detailed display of the SL-scores (color-coded) of the treatment given in the trial (bottom row) and the SL-scores of all candidate therapies (all other rows) for all 71 patients in the trial (the treatments considered are denoted in every column). Blue boxes denote the best treatments (with highest SL-scores) recommended for each patient. Cancer types of each sample are color-coded at the bottom of the figure. In particular, computation of the SL-scores for each of the drugs in every patient based on its tumor transcriptomics is conducted. The resulting analysis shows that for approximately 92% (66/71) of the patients, alternate therapies that have higher SL-scores than the drugs prescribed to them in the trial could have been identified. Based on the 0.44 optimal classification threshold identified above, 70% (50/71) of the patients are predicted to respond to the new treatments, compared to 26% that responded (based on either targeted DNA sequencing or transcriptomics) in the original trial. Of the 52 non-responders reported in the trial, 69% (36/52) of the patients can be matched with predicted effective therapies (with 5% false positive rate).

To illustrate the potential future application of SELECT for patient stratification, we describe here two individual cases arising in the trial data analysis. The first involves an 82-year-old male neuroendocrine cancer patient who was treated with everolimus because of an PIK3CA overexpression, and the patient indeed responded to the therapy. SELECT also recommends the treatment of everolimus, as shown in FIG. 4E. The second example involves a 75-year-old male colon cancer patient who was treated with cabozantinib in the trial because of VEGFA and HGF overexpression but failed to respond to the therapy. SELECT assigns a very low SL score to cabozantinib but suggests alternative therapies that obtain much higher SL scores, as shown in FIG. 4F. Overall, the drugs most frequently recommended by SELECT include a multi-tyrosine kinase inhibitor (pazopanib) followed by a cell cycle checkpoint inhibitor (palbociclib) and an EGFR inhibitor.

Samples that display a strong SL vulnerability to one drug tend to have SL-mediated vulnerabilities to many other targeted agents, indicating that SL-based treatment opportunities may actually increase in advanced tumors. Reassuringly, an SL-based drug coverage analysis in another independent transcriptomics-based trials dataset from the Tempus cohort, focusing on the same cancer types and drugs as those studied in the trial, shows a similar pattern of top recommended drugs (as shown in FIG. 4H), pointing to the robustness of these predictions across a variety of patient cohorts

In addition to the trial cohorts, we analyzed the recently released POG570 cohort, where the post-treatment transcriptomics data together with treatment history is available for advanced or metastatic tumors of 570 patients. We first confirmed that the samples SL-scores are associated with longer treatment duration, which served as a proxy for therapeutic response in the original publication (shown in FIG. 4I). We further confirmed that this trend holds true per individual drugs (FIG. 4J) and across individual cancer types (FIG. 4K).

Finally, we asked whether SELECT can successfully estimate the objective response rates (ORR) observed across different drug treatments in different clinical trials for a given cancer type. As these trials did measure and report the patients' tumor transcriptomics, we estimated, for each drug, its coverage (the patients who are predicted to respond based on their SL scores being larger than the 0.44 response threshold) in the TCGA cohort of the relevant cancer type (Methods). We collected ORR data from multiple clinical trials in melanoma and non-small cell lung cancer (a total of 3,246 patients from 18 trials). Reassuringly, we find that the resulting estimated coverage is significantly correlated with the observed ORR in both cancer types.

FIG. 5 is a flowchart of a method 500 for predicting survival rates in subjects or populations affected by a disease or disorder. In one particular implementation, the method 500 may be executed to identify a corresponding cancer therapy based on a transcriptomic profile of a tumor of a patient. In some instances, the operations of the method 500 may be performed by a computing device executing code or other software, such as the computing device described in more detail below. Further, the operations may be executed via one or more hardware components, execution of one or more programs, or a combination of both hardware components and software programs.

Beginning in operation 502, the method 500 may obtain, from one or more databases storing genetic interaction information, a plurality of candidate synthetic lethality (SL) gene partners for a cancer therapy. The one or more databases may store any number of SL gene partners for different cancer therapies and may be accessible by the computing device via a network or may be directly connected to the computing device. In another embodiment, the one or more databases may also or separately store candidate synthetic rescuer (SR) gene partners for a cancer therapy and such SR gene partners may also or separately be obtained. In operation 504, the method 500 may identify a subset of the obtained candidate SL gene partners based on patient response data and phylogenetic profile information of each of the plurality of candidate SL gene partners. The patient response data and/or phylogenetic profile information may be obtained from a separate database, the same database, or may be calculated by a computing device executing the method 500. In an alternate implementation, a subset of candidate SR gene partners may be identified as potential predictive biomarkers for the cancer therapy. The identification of the biomarkers may be based on at least one of (1) a product of PD1 and PDL1 gene expression levels, (2) CTLA4 protein expression levels, and (3) gene expression levels and somatic copy number alterations (SCNA) of each of a plurality of candidate SR gene partners.

In operation 506, the subset of the plurality of candidate SL gene partners may be filtered via a comparison to a BRAF inhibitor dataset, also obtained by a computing device from the database or a separate database. The comparison may provide an SL-score for each of the subset of the plurality of candidate SL gene partners such that the subset may be ranked based on the SL-score. In the alternate embodiment in which SR gene partners are considered, the method may rank the subset of the plurality of candidate SR gene partners based on phylogenetic distance information to filter the subset of the plurality of candidate SL gene partners and filter the ranked subset of the plurality of candidate SR gene partners based on an identification of candidate SR gene partners in which a downregulation (or upregulation) of a partner rescuer gene occurs. Finally, in operation 508, a cancer therapy for a patient may be identified, based at least on a transcriptomics profile of a tumor of the patient and the filtered subset of the plurality of candidate SL gene partners or SR gene partners, the cancer therapy for the patient. As such, through the method 500 of FIG. 5, a prediction of the likelihood of a subject to respond to a therapy for treatment of the disease or disorder and/or predict improved therapies for treatment of the disease or disorder may be made and a corresponding therapy may be selected for the patient.

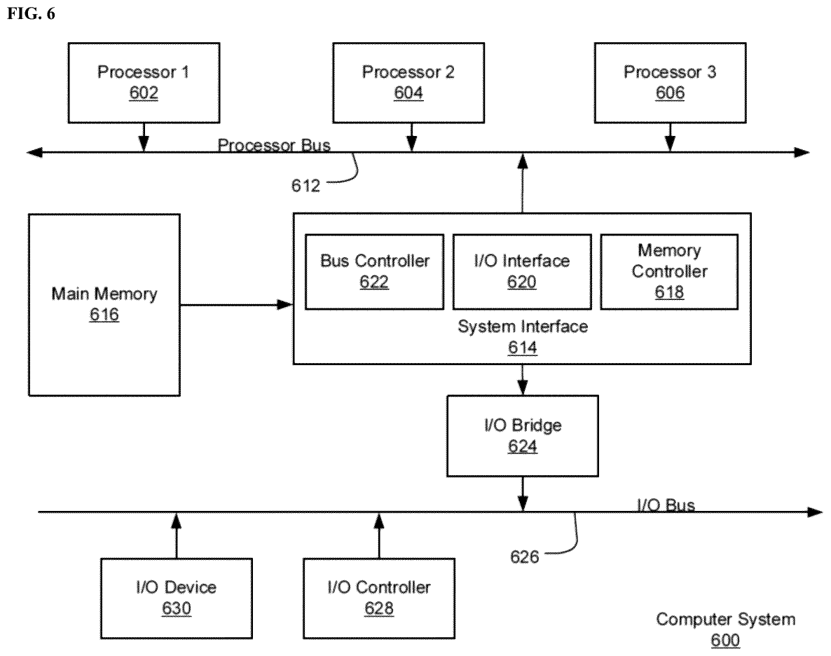

FIG. 6 is a block diagram illustrating an example of a computing device or computer system 600 which may be used in implementing the embodiments of the components of the network disclosed above. For example, the computing system 600 of FIG. 6 may perform one or more of the operations discussed above. The computer system (system) includes one or more processors 602-606. Processors 602-606 may include one or more internal levels of cache (not shown) and a bus controller or bus interface unit to direct interaction with the processor bus 612. Processor bus 612, also known as the host bus or the front side bus, may be used to couple the processors 602-606 with the system interface 614. System interface 614 may be connected to the processor bus 612 to interface other components of the system 600 with the processor bus 612. For example, system interface 614 may include a memory controller 614 for interfacing a main memory 616 with the processor bus 612. The main memory 616 typically includes one or more memory cards and a control circuit (not shown). System interface 614 may also include an input/output (I/O) interface 620 to interface one or more I/O bridges or I/O devices with the processor bus 612. One or more I/O controllers and/or I/O devices may be connected with the I/O bus 626, such as I/O controller 628 and I/O device 640, as illustrated.

I/O device 640 may also include an input device (not shown), such as an alphanumeric input device, including alphanumeric and other keys for communicating information and/or command selections to the processors 602-606. Another type of user input device includes cursor control, such as a mouse, a trackball, or cursor direction keys for communicating direction information and command selections to the processors 602-606 and for controlling cursor movement on the display device.

System 600 may include a dynamic storage device, referred to as main memory 616, or a random access memory (RAM) or other computer-readable devices coupled to the processor bus 612 for storing information and instructions to be executed by the processors 602-606. Main memory 616 also may be used for storing temporary variables or other intermediate information during execution of instructions by the processors 602-606. System 600 may include a read only memory (ROM) and/or other static storage device coupled to the processor bus 612 for storing static information and instructions for the processors 602-606. The system set forth in FIG. 6 is but one possible example of a computer system that may employ or be configured in accordance with aspects of the present disclosure.

According to one embodiment, the above techniques may be performed by computer system 600 in response to processor 604 executing one or more sequences of one or more instructions contained in main memory 616. These instructions may be read into main memory 616 from another machine-readable medium, such as a storage device. Execution of the sequences of instructions contained in main memory 616 may cause processors 602-606 to perform the process steps described herein. In alternative embodiments, circuitry may be used in place of or in combination with the software instructions. Thus, embodiments of the present disclosure may include both hardware and software components.

A machine readable medium includes any mechanism for storing or transmitting information in a form (e.g., software, processing application) readable by a machine (e.g., a computer). Such media may take the form of, but is not limited to, non-volatile media and volatile media. Non-volatile media includes optical or magnetic disks. Volatile media includes dynamic memory, such as main memory 616. Common forms of machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

What is claimed is:

1. A method of identifying a cancer therapy for a patient wherein the cancer therapy for the patient is identified by:
accessing one or more databases storing information associated with genetic interactions to obtain a plurality of candidate synthetic lethality (SL) gene partners for a cancer therapy;
identifying, based on experimental functional screens, reference patients' omics and survival data and phylogenetic profile information of each of the plurality of candidate SL gene partners, a subset of the plurality of candidate SL gene partners as potential predictive biomarkers for the cancer therapy;
comparing the subset of the plurality of candidate SL gene partners to a cancer-inhibiting drug dataset to filter the subset of the plurality of candidate SL gene partners; and
identifying, based on a transcriptomics profile of a tumor of the patient and the filtered subset of the plurality of candidate SL gene partners, the cancer therapy for the patient.

2. A method of identifying a cancer therapy for a patient, the method comprising:
accessing one or more databases storing genetic interaction information to obtain a plurality of candidate synthetic rescue (SR) gene partners for a cancer therapy;
identifying, based on at least one of (1) a product of PD1 and PDL1 activity, (2) CTLA4 activity, and (3) molecular profiles including gene expression levels and somatic copy number alterations (SCNA) of each of a plurality of candidate SR gene partners, a subset of candidate SR gene partners as potential predictive biomarkers for the cancer therapy;
ranking the subset of the plurality of candidate SR gene partners based on phylogenetic distance information to filter the subset of the plurality of candidate SR gene partners;
filtering, based on an identification of candidate SR gene partners in which a downregulation (or upregulation) of a partner rescuer gene occurs, the ranked subset of the plurality of candidate SR gene partners; and,
identifying, based on a transcriptomics profile of a tumor of the patient and the filtered subset of the plurality of candidate SR gene partners, a cancer therapy for the patient.

3. A method of treating a cancer patient, comprising administering a cancer therapy to a patient in need thereof, wherein the cancer therapy was identified according to the method of claim 1 or claim 2.

4. The method of claim 1 further comprising:
assigning a score to each of the plurality of candidate SL gene partners based on patient response data to each of the plurality of candidate SL gene partners; and
filtering, based on the assigned scores, the plurality of candidate SL gene partners to identify the subset the plurality of candidate SL gene partners.

5. The method of claim 4 further comprising:
ranking, based on the assigned scores, the plurality of candidate SL gene partners, wherein the subset of the plurality of candidate SL gene partners comprises a subset of the plurality of candidate SL gene partners with the highest assigned scores.

6. The method of claim 5 wherein the subset of the plurality of candidate SL gene partners comprises 25 gene partners.

7. The method of claim 2 wherein the subset of the plurality of candidate SR gene partners comprises 10 gene partners.

8. The method of claim 1 wherein the transcriptomics profile comprises at least one of a proliferation measurement value, a cytolytic value, or a target gene expression identification level.

9. A system for identifying a cancer therapy for a patient, the system comprising:
a processor; and
a tangible storage medium storing instructions that are executed by the processor to perform operations comprising:
accessing one or more databases storing genetic interaction information to obtain a plurality of candidate synthetic lethality (SL) gene partners for a cancer therapy;
identifying, based on experimental functional screens, reference patients' omics and survival data and phylogenetic profile information of each of the plurality of candidate SL gene partners, a subset of the plurality of candidate SL gene partners as potential predictive biomarkers for the cancer therapy;
comparing the subset of the plurality of candidate SL gene partners to a cancer-inhibiting drug dataset to filter the subset of the plurality of candidate SL gene partners and,
identifying, based on a transcriptomics profile of a tumor of the patient and the filtered subset of the plurality of candidate SL gene partners, the cancer therapy for the patient.

10. A system for identifying a cancer therapy for a patient, the system comprising:
a processor; and
a tangible storage medium storing instructions that are executed by the processor to perform operations comprising:
accessing one or more databases storing information related to genetic interactions to obtain a plurality of candidate synthetic rescue (SR) gene partners for a cancer therapy;
identifying, based on reference patients' omics and survival data and phylogenetic profile information comprising at least one of (1) a product of PD1 and PDL1 gene expression levels, (2) CTLA4 protein expression levels, and (3) a plurality of candidate SR gene partners, a subset of candidate SR gene partners as potential predictive biomarkers for the cancer therapy;
ranking the subset of the plurality of candidate SR gene partners based on phylogenetic distance information to filter the subset of the plurality of candidate SR gene partners;
filtering, based on an identification of candidate SR gene partners in which a downregulation (or upregulation) of a partner rescuer gene occurs, the ranked subset of the plurality of candidate SR gene partners; and,
identifying, based on a transcriptomics profile of a tumor of the patient and the filtered subset of the plurality of candidate SR gene partners, a cancer therapy for the patient.

11. A method of treating a cancer in a subject in need thereof, comprising administering an anti-cancer therapy listed in Table 1 to the subject, wherein a sample of the cancer has been determined to have a Synthetic Lethality (SL)-score >0.44, wherein the SL-score was determined by:
a. providing expression levels of SL partner genes in the subject cancer sample, wherein the SL partner genes comprise a plurality of genes associated with the anti-cancer therapy in Table 1;
b. providing expression levels of the SL partner genes in each of a plurality of reference cancer samples;
c. counting the number of the SL partner genes from the subject cancer sample that are downregulated compared to expression levels of the respective SL partner genes among the reference cancer samples; and,
d. dividing the number counted in (c) by the total number of the SL partner genes;
wherein a SL-score >0.44 indicates that the subject's cancer is sensitive to the anti-cancer therapy.

12. The method of claim 11, wherein the SL partner genes in steps (c) and (d) consist of the genes associated with the anti-cancer therapy in Table 1.

13. The method of claim 11 or claim 12, wherein a SL partner gene in the subject cancer sample is downregulated if the expression level of the SL partner gene in the subject cancer sample is in the bottom tertile of expression levels of the respective SL partner gene among the reference cancer samples.

14. The method of any one of claims 11-13, wherein the subject's cancer and the reference cancer samples are of the same type of cancer.

15. The method of any one of claims 11-14, wherein the SL partner gene expression levels are measured from RNA-sequencing (RNAseq) or microarray data.

16. The method of any one of claims 11-15, wherein the SL partner gene expression levels are normalized.

17. The method of claim 16, wherein the SL partner gene expression levels are measured from RNAseq and the normalization method is Reads per Kilobase per Million mapped reads (RPKM)/RNAseq by Expectation-Maximization (RSEM).

18. The method of any one of claims 11-17, wherein the SL partner gene expression levels of the reference cancer samples are from the Cancer Genome Atlas (TCGA).

19. The method of any one of claims 11-18, wherein the anti-cancer therapy comprises Vemurafenib, and the SL partner genes comprise FSCN1, ICA1, PMS2, C1GALT1, MMD2, C7orf28B, NT5C3, NDUFA4, RAPGEF5, TMEM106B, ADCYAP1R1, SCIN, NEUROD6, RP9, FAM126A, KLHL7, SKAP2, TRA2A, JAZF1, CBX3, BBS9, SP8, MACC1, GGCT, and TAX1BP1.

20. The method of any one of claims 11-18, wherein the anti-cancer therapy comprises Tamoxifen, and the SL partner genes comprise LTBP2, GADL1, CRISP2, SLC13A5, PCDHGA7, NLRP10, AAK1, IL22RA2, RASGRF1, FAM19A3, TPM2, UBR4, LRRFIP1, FOXL1, PCDHGA2, MAMSTR, ABCG4, FBXO32, DSG3, FER, ALPP, PINX1, AVPR1A, LHX6, and PHLPP2.

21. The method of any one of claims 11-18, wherein the anti-cancer therapy comprises Anthracycline, and the SL partner genes comprise NFKB1, ZNF667, NMNAT1, DAPK1, ELOVL7, TINAGL1, PCDHGA7, ZNF470, PIWIL4, ZNF471, ZNF300, GALNTL2, CPM, ECHDC3, RBM7, POU2F2, ARHGAP6, H6PD, EIF4G3, NCF4, SH3GLB1, AADAC, SLC25A24, STX11, and ADAMTS5.

22. The method of any one of claims 11-18, wherein the anti-cancer therapy comprises Lapatinib, Epirubicin, and Fluorouracil, and the SL partner genes comprise SLC7A6, NFKB1, ZNF667, ELOVL7, TINAGL1, LTBP4, AP4E1, PCDHGA7, PIWIL4, KIF3C, ZNF471, AADACL2, CPM, ECHDC3, RBM7, POU2F2, STXBP1, RPL4, TOMM40L, H6PD, ALS2, AMMECR1L, PACS1, CSMD3, and RLBP1.

23. The method of any one of claims 11-18, wherein the anti-cancer therapy comprises Anastrozole, and the SL partner genes comprise MMP21, ZNF662, ANKRD1, ALPP, H2AFY2, FGF10, DCAF12L1, KIAA1549, RBM12, PTPRN2, LRRC8A, TSPAN14, MYH11, SLC4A7, HECW2, NKD1, ARHGDIG, RC3H2, MLL3, DRD1, TAF1L, SLC7A6, ZXDB, VSIG2, and TMEFF2.

24. The method of any one of claims 11-18, wherein the anti-cancer therapy comprises Bortezomib, Thalidomide, and Dexamethasone, and the SL partner genes comprise ALPP, LCTL, LTA4H, BMP8B, CPN2, NAALAD2, KRT16, ZNF600, IGF2BP3, RPP25, ACOT8, TNNC1, CGB, PTGES, SLC17A7, ROS1, EPHA8, ABCG4, CYP11A1, PKP3, MYL6, STAU1, F3, LTBP4, and FGF23.

25. The method of any one of claims 11-18, wherein the anti-cancer therapy comprises Irinotecan and Fluorouracil, and the SL partner genes comprise MTRF1L, MAPK11, TXK, RPL4, NADK, SLC7A6, ACOX2, SOD2, STXBP1, SLC9A8, LIMK2, ARHGDIG, DIRAS1, SLC2A5, DUSP7, RPL5, STK32A, CCND3, SOD2, ECHDC3, CSNK1A1, PCSK7, CABP4, KIF1B, and LDHAL6B.

26. The method of any one of claims 11-18, wherein the anti-cancer therapy comprises Doxorubicin and Gemcitabine, and the SL partner genes comprise NFKB1, ZNF667, NMNAT1, DAPK1, ELOVL7, SMAP2, TINAGL1, PCDHGA7, ZNF470, PIWIL4, ZNF471, ERI1, KCNH4, AADACL2, RPL11, RFX2, CPM, ECHDC3, RBM7, POU2F2, STXBP1, AGTPBP1, SLC6A16, CFI, and ARHGAP6.

27. The method of any one of claims 11-18, wherein the anti-cancer therapy comprises Folinic Acid (FA), Irinotecan, and Fluorouracil, and the SL partner genes comprise FAM83G, DNAJC12, RALBP1, TXK, RPL4, NADK, MYBPC2, AGTR2, SLC7A6, ACOX2, SOD2, STXBP1, LIMK2, ARHGDIG, DIRAS1, POLA2, SLC2A5, SLC9A9, PMP22, SOD2, ECHDC3, KIRREL, NTAN1, CABP4, and KIF1B.

28. The method of any one of claims 11-18, wherein the anti-cancer therapy comprises Afatinib, and the SL partner genes comprise CHCHD3, SLC7A6, EPHB2, CFL1, SLCO4A1, PAX3, MORC2, NROB1, OR1F1, RADIL, KCNH2, LTBP4, ADCY3, KIF3C, TMSB10, STYXL1, TAGLN2, NFKBIL2, PUSL1, JAM3, TRIP13, PTDSS1, QSOX1, CDCA7L, and APCDD1L.

29. The method of any one of claims 11-18, wherein the anti-cancer therapy comprises Azacitidine, and the SL partner genes comprise PCDHGA7, MLLT4, CHD5, WFIKKN2, DRD1, BACH1, TUB, PDZD4, CLDN18, PCDHGA10, PCDHGA6, BAZ2A, KIAA0355, ESYT2, PRSS33, ZNF300, CAPS2, RHOBTB3, PCDHGA5, KDM6B, PRDM2, ZNF573, GPR114, AADAC, and ANKRD17.

30. The method of any one of claims 11-18, wherein the anti-cancer therapy comprises Belinostat, and the SL partner genes comprise SOD2, TTPAL, VPS26A, AURKC, KLF17, CYTH3, EIF1, CTH, TSC22D2, RER1, AGPAT4, ATP5B, DACT1, RNF24, HIST1H2AL, CEBPB, MFI2, DDX19B, VAMP3, GNB1, PGK2, GFRA2, NR5A1, RNF24, and MAN2A1.

31. The method of any one of claims 11-18, wherein the anti-cancer therapy comprises Bortezomib, and the SL partner genes comprise LTA4H, TNNC1, MYL6, RPS12, MYL6, PROSC, ORMDL2, CAPZB, VAMP3, RPS8, SOD2, PGM1, GPX7, PRDX6, DDX47, CSNK2A2, PGM1, SFN, CPS1, KPNA6, KARS, AKT2, MRTO4, EIF3I, and ARL4C.

32. The method of any one of claims 11-18, wherein the anti-cancer therapy comprises Cabozantinib, and the SL partner genes comprise TOMM40L, LAMB4, NUCB1, PCDHB13, SERTAD1, FBXL22, C2orf24, INPP5A, C14orf138, USP35, BLCAP, NIPSNAP3A, TACR2, ZNF257, EIF2B3, WNT10A, ZYX, PCDH8, KDM4B, KCNJ14, LGALS7, AVEN, TMEM18, TNC, and NRBF2.

33. The method of any one of claims 11-18, wherein the anti-cancer therapy comprises Carfilzomib, and the SL partner genes comprise RPS12, SRF, BIN3, PSMD7, PINX1, MSRA, FDFT1, ERI1, LONRF1, PCDHAC1, SOX7, UBA7, TLE4, PPP1R3B, MTMR9, TMEM110, GATA4, SLC7A2, RAB27A, NEFM, CTSB, PRPF38A, KCNAB2, PAQR5, and MBD3L1.

34. The method of any one of claims 11-18, wherein the anti-cancer therapy comprises Cetuximab, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

35. The method of any one of claims 11-18, wherein the anti-cancer therapy comprises Cobimetinib, and the SL partner genes comprise CPT2, RPL5, CTDSPL2, MAP1LC3A, HBS1L, USP3, MRTO4, ELOVL6, PRKACG, TADA2A, CUL5, ME3, STXBP3, DHX35, CSNK1G3, RBBP4, MRE11A, NAA16, BCAT2, RAB33B, DDX46, CABP5, WDR26, LACTB2, and PRPSAP1.

36. The method of any one of claims 11-18, wherein the anti-cancer therapy comprises Crizotinib, and the SL partner genes comprise TOMM40L, PCDHB13, FBXL22, C2orf24, C1GALT1, INPP5A, USP35, TACR2, ZNF257, KDM4B, TMEM18, ACOX2, RXRG, BMP8B, AZU1, FAM36A, NAT15, PLA2G3, C10orf140, PHYH, SS18, BRMS1L, C17orf61, PHTF1, and HNRNPH3.

37. The method of any one of claims 11-18, wherein the anti-cancer therapy comprises Denileukin, and the SL partner genes comprise PSAPL1, CNTF, MTL5, ARTS, ZNF300, TACR2, EPHX3, WBP11P1, CCK, SKP2, EXD1, NFE2L3, B3GALT5, NEU4, FAM3B, NPPC, KLHL34, HUWE1, TREML2, GAL, C12orf48, C2orf43, ZNF829, HBQ1, and FAM105B.

38. The method of any one of claims 11-18, wherein the anti-cancer therapy comprises Dinutuximab, and the SL partner genes comprise ZIK1, RFX2, RNF24, CDKL5, FAM179B, NCOA3, GPRC5C, SPINK2, DNAJB12, S100B, CIDEA, CYP11A1, TBC1D2, CLMN, SLC16A12, NPTX2, SHANK2, CEP164, DAAM1, MMP15, MAST4, MAPK4, GPRIN2, MT1A, and IL18.

39. The method of any one of claims 11-18, wherein the anti-cancer therapy comprises Durvalumab, and the SL partner genes comprise HMGA1, E2F2, TMEM69, C20orf20, PHLDA2, BIRC7, PSAPL1, C1orf135, POLQ, IQGAP3, PRAME, KIAA1524, ZNF598, ZNF695, ZNF581, HTATIP2, SLCO4A1, TMPRSS13, KIF23, LMNB2, CCNA2, BUB1, PSRC1, SLC16A1, and KIFC1.

40. The method of any one of claims 11-18, wherein the anti-cancer therapy comprises Estramustine, and the SL partner genes comprise MEA1, PKHD1, MRPS10, BMP8B, SSB, LMLN, ZNF829, HSD11B1, STXBP5, ZNF239, NCDN, GLIS2, QSOX1, RBMS3, KRT75, MASTL, ITGA11, C1QC, TMC7, MS4A8B, ADCY7, FGF5, MLF1, KIF4B, and PLP2.

41. The method of any one of claims 11-18, wherein the anti-cancer therapy comprises Everolimus, and the SL partner genes comprise PRKCG, DNAJB12, CDK16, C20orf118, STK4, CTH, NOX1, DNAJC2, FGR, TAF12, DDOST, RPS6KA1, TSSK3, RHEBL1, SLC7A6, MYLK4, KMO, PSMB9, HERC4, SGPL1, PTPN6, SLC2A5, NOL10, TRNAU1AP, and VAC14.

42. The method of any one of claims 11-18, wherein the anti-cancer therapy comprises Gemtuzumab, and the SL partner genes comprise WNT10B, TAF4, MFSD2B, PTGDS, SPRR3, GATAD2A, OPRL1, ALOX15, MESP2, PSAPL1, B3GALT5, LRRC42, BIRC7, SLC4A2, PLEKHA6, NR5A1, SOX10, HNRNPL, KLHL30, GALK1, RHPN2, STAU1, DBNDD1, UPK1A, and NCDN.

43. The method of any one of claims 11-18, wherein the anti-cancer therapy comprises Ibrutinib, and the SL partner genes comprise SLC6A14, CFL1, ABCC10, CORO1B, OPRL1, HORMAD1, RCE1, ZNF239, ZDHHC7, GPRC5D, KRT78, KRT6C, RGS1, PADI3, UNC13D, B3GALT5, WNT10B, C1GALT1, HSD17B2, RGS19, MARK2, NAA40, SLC7A6, DBNL, and UPP1.

44. The method of any one of claims 11-18, wherein the anti-cancer therapy comprises Idelalisib, and the SL partner genes comprise MFSD2B, POLQ, TXK, CELA3B, ZC3H12A, GNL3, ATAD5, DNAJC2, LARP1, C1GALT1, TAS1R1, RPRD1B, HDLBP, KRT75, STK4, CCDCl9, NOL10, BHLHE40, RNF24, CNNM4, SLC7A5, CLSPN, CREG2, LRRC42, and MYC.

45. The method of any one of claims 11-18, wherein the anti-cancer therapy comprises Lapatinib, and the SL partner genes comprise SLC7A6, B4GALT5, PACS1, CSMD3, VPS13B, CHRFAM7A, PGCP, MTBP, RIMS2, TMEM74, NIPAL2, C8orf37, RNF19A, ADCY3, FBXO43, SNX31, KCNS2, HAS2, KIAA0196, STX16, DOK5, UTP23, CDH17, DERL1, and TTPAL.

46. The method of any one of claims 11-18, wherein the anti-cancer therapy comprises Lenvatinib, and the SL partner genes comprise PIGT, KNTC1, SNX5, LEP, TAX1BP3, FAM83D, ERGIC1, FGF5, LRRC42, MYC, KCNA7, HOXC8, CDC25B, P4HA3, KIF3C, GREM1, CDC25B, CDC25B, LAMB1, FLNC, FHOD1, SLC7A6, ZFHX4, ITGA5, and NUMBL.

47. The method of any one of claims 11-18, wherein the anti-cancer therapy comprises Midostaurin, and the SL partner genes comprise FLNC, COTL1, CPA4, GREM1, HOXC5, IGF2BP1, IGF2BP3, PDLIM7, SKP2, HOXC8, YBX1, HNRNPL, DPH2, C15orf42, EPHB2, HTR1D, ZDHHC7, NADK, CTHRC1, DHX34, CPXM1, TPX2, DCLRE1B, CDCA7, and PDLIM3.

48. The method of any one of claims 11-18, wherein the anti-cancer therapy comprises Nintedanib, and the SL partner genes comprise SND1, PIGT, CHD5, PLXNA3, SEH1L, HNRNPA1L2, IGLON5, SNX5, TRIP13, TRIM71, TAX1BP3, KCNQ2, LHX6, ERGIC1, TES, PTDSS2, LLPH, PCDH8, C1GALT1, RADIL, PSMD8, C3orf26, TRPC4AP, BCAP31, and TMED3.

49. The method of any one of claims 11-18, wherein the anti-cancer therapy comprises Olaratumab, and the SL partner genes comprise UBQLN4, LRRIQ4, TAF4, PCDHB13, WWC1, HNRNPL, HPDL, ANKRD2, EFHD1, ZNF236, PTCD1, MRPS18A, WDR93, SLC7A6, GSTM3, CXorf40B, ERGIC1, COBL, DHX34, CHD5, SP9, MTPAP, PKP2, HSD1162, and SPATA2.

50. The method of any one of claims 11-18, wherein the anti-cancer therapy comprises Palbociclib, and the SL partner genes comprise TPT1, RPL34, RPS6KA6, CSNK1G3, ATP13A4, ACSS2, IMMP2L, PMPCB, RDH13, ATP13A5, ADI1, SUCLA2, MAN1C1, ARL6, MAP1LC3A, SLC9A9, EIF3D, PLS1, EIF1, UCP1, ACSS3, AHCYL1, MOBKL2C, RPL4, and RPS20.

51. The method of any one of claims 11-18, wherein the anti-cancer therapy comprises Pexidartinib, and the SL partner genes comprise MFSD2B, TAF4, BIRC7, ALPPL2, C1orf135, LRRC42, HNRNPC, C1GALT1, BMP8B, MSLN, HIST1H1D, POLQ, SLC35A2, RGS19, LHX2, HNRNPUL1, PPP1R3G, LRFN4, WNT10B, GREM1, CCNA2, KIF2C, CSTB, RPRD1B, and CCNF.

52. The method of any one of claims 11-18, wherein the anti-cancer therapy comprises Pomalidomide, and the SL partner genes comprise HN1L, CCNF, MNS1, HNRNPAB, KRT75, AIMP2, SFPQ, KRT6B, HPDL, TRIP13, DPF1, CHAF1B, KIF2C, DMBX1, NCAPD2, HK1, PPM1G, CCNA2, SKA1, PAICS, KIF23, RADIL, GREM1, SV2B, and SLC7A5.

53. The method of any one of claims 11-18, wherein the anti-cancer therapy comprises Ponatinib, and the SL partner genes comprise SND1, FLNC, CHD5, SEH1L, CTRB2, HNRNPA1L2, IGLON5, ANKRD40, KCNK9, TSPAN15, SNX5, TRIP13, KRT3, TRIM71, CCRN4L, PSMG2, KCNQ2, ATP5G3, LHX6, ERGIC1, TES, PTDSS2, PCDH8, CRCP, and PSMD8.

54. The method of any one of claims 11-18, wherein the anti-cancer therapy comprises Porfimer, and the SL partner genes comprise FAM83B, KLF14, KLK8, CALHM3, HOXB13, GMNN, B3GALT5, NRG3, PSAPL1, GSTP1, SLCO4A1, CGREF1, DLX2, C2orf39, FAM63B, S100A7, PSG4, TNNT3, KRT6A, TET1, GPR64, SPRR3, MITF, MNT, and DIO3.

55. The method of any one of claims 11-18, wherein the anti-cancer therapy comprises Prexasertib, and the SL partner genes comprise FBX040, CELF4, PCDHGA7, ERP44, GATA5, NUP214, HGSNAT, XKR6, BACH1, CCBP2, PCDHA11, SLC6A3, ZNF300, KLK1, TMEM90A, SLC7A2, IQGAP1, ZNF560, SFTPA1, ECHDC3, MITF, ANKDD1A, PCDHGA3, TLE4, and AGPAT9.

56. The method of any one of claims 11-18, wherein the anti-cancer therapy comprises Regorafenib, and the SL partner genes comprise SPATA2, PTCD1, GSTM3, CSTB, PCSK6, DLL1, GALNS, SLC4A2, PIGT, CCAR1, NPBWR1, SFPQ, ZNF283, TNC, PTPN2, DLG5, USP49, SRRM1, LCTL, SEH1L, NFYA, CTRB2, GRID2IP, USP35, and MMP21.

57. The method of any one of claims 11-18, wherein the anti-cancer therapy comprises Romidepsin, and the SL partner genes comprise SOD2, TTPAL, VPS26A, AURKC, NID1, KLF17, EIF1, CTH, UBE2D3, TSC22D2, RER1, AGPAT4, ATPSB, DACT1, RNF24, HIST1H2AL, CEBPB, PARVB, PLIN2, DDX19B, VAMP3, PGK2, ACTR2, GNB1, and RHOC.

58. The method of any one of claims 11-18, wherein the anti-cancer therapy comprises Ruxolitinib, and the SL partner genes comprise PLXNA3, CCNB2, IQGAP3, PADI3, C20orf20, KIF23, IGF2BP3, TIPIN, CNPY3, FOXM1, KIAA1524, ZDHHC7, MFSD2B, HOXB13, IQGAP3, TPX2, SMC4, XRCC2, PHLDA2, NSUN2, NCAPG2, POLQ, KDMSC, SLC4A2, and MYBL2.

59. The method of any one of claims 11-18, wherein the anti-cancer therapy comprises Siltuximab, and the SL partner genes comprise POMC, ZYG11A, PARP10, DENND3, RBM15, KLK8, BIRCS, APOO, INSM2, MT3, AMELX, CCR1, C13orf36, TRIM48, PDCD7, MLL3, MT1F, LCN15, HES4, AIRE, ZC3H18, TAF3, OR2L13, NCR2, and DIAPH2.

60. The method of any one of claims 11-18, wherein the anti-cancer therapy comprises Sonidegib, and the SL partner genes comprise ADAMTS14, TTLL12, CREB3L1, TMC4, NBEAL2, BMP8B, ERGIC1, SRPX2, P4HA3, AVPR1A, CCND1, PLEKHF1, NGF, COL10A1, LDLRAP1, PROM2, TAGLN2, FOXL1, TFAP2A, PITRM1, UNC5A, ANO1, HTATIP2, NAGA, and SPRY4.

61. The method of any one of claims 11-18, wherein the anti-cancer therapy comprises Sorafenib, and the SL partner genes comprise SPATA2, PTCD1, GSTM3, DHX34, CSTB, PCSK6, FLNC, DLL1, GALNS, SLC4A2, PIGT, CCAR1, NPBWR1, SFPQ, ZNF283, TNC, CCDC112, PTPN2, DLG5, USP49, SRRM1, LCTL, SEH1L, NFYA, and CTRB2.

62. The method of any one of claims 11-18, wherein the anti-cancer therapy comprises Sunitinib, and the SL partner genes comprise SPATA2, PTCD1, GSTM3, DHX34, PCSK6, FLNC, DLL1, MFSD2B, SLC4A2, PIGT, CCAR1, NPBWR1, SFPQ, ZNF283, CCDC112, DLG5, USP49, SRRM1, NFYA, GRID2IP, USP35, TAF4, MMP21, HPDL, and OR10H1.

63. The method of any one of claims 11-18, wherein the anti-cancer therapy comprises Vandetanib, and the SL partner genes comprise LAMB4, NUCB1, CABP4, SERTAD1, BLCAP, EIF2B3, ZYX, PCDH8, KCNJ14, AVEN, TNC, NRBF2, TOMM40L, RFPL4B, RADIL, FBXL13, DNAJC8, ZNF701, MYCBP, ZNF581, ADAM30, LRRC39, SUSD1, CRISPLD2, and ATP5SL.

64. The method of any one of claims 11-18, wherein the anti-cancer therapy comprises Vorinostat, and the SL partner genes comprise SOD2, TTPAL, VPS26A, AURKC, NID1, KLF17, EIF1, CTH, TSC22D2, RER1, AGPAT4, ATP5B, DACT1, RER1, RNF24, HIST1H2AL, CEBPB, PARVB, DDX19B, VAMP3, GNB1, PGK2, ADH1B, ACTR2, and GNB1.

65. The method of any one of claims 11-18, wherein the anti-cancer therapy comprises Exemestane, and the SL partner genes comprise MMP21, ZNF662, ANKRD1, ALPP, H2AFY2, FGF10, DCAF12L1, KIAA1549, RBM12, PTPRN2, LRRC8A, TSPAN14, MYH11, SLC4A7, HECW2, NKD1, ARHGDIG, RC3H2, MLL3, DRD1, TAF1L, SLC7A6, ZXDB, VSIG2, and TMEFF2.

66. The method of any one of claims 11-18, wherein the anti-cancer therapy comprises Letrozole, and the SL partner genes comprise MMP21, ZNF662, ANKRD1, ALPP, H2AFY2, FGF10, DCAF12L1, KIAA1549, RBM12, PTPRN2, LRRC8A, TSPAN14, MYH11, SLC4A7, HECW2, NKD1, ARHGDIG, RC3H2, MLL3, DRD1, TAF1L, SLC7A6, ZXDB, VSIG2, and TMEFF2.

67. The method of any one of claims 11-18, wherein the anti-cancer therapy comprises Decitabine, and the SL partner genes comprise PCDHGA7, MLLT4, CHD5, WFIKKN2, DRD1, BACH1, TUB, PDZD4, CLDN18, PCDHGA10, PCDHGA6, BAZ2A, KIAA0355, ESYT2, PRSS33, ZNF300, CAPS2, RHOBTB3, PCDHGA5, KDM6B, PRDM2, ZNF573, GPR114, AADAC, and ANKRD17.

68. The method of any one of claims 11-18, wherein the anti-cancer therapy comprises Panobinostat, and the SL partner genes comprise SOD2, TTPAL, VPS26A, AURKC, KLF17, CYTH3, EIF1, CTH, TSC22D2, RER1, AGPAT4, ATP5B, DACT1, RNF24, HIST1H2AL, CEBPB, MFI2, DDX19B, VAMP3, GNB1, PGK2, GFRA2, NR5A1, RNF24, and MAN2A1.

69. The method of any one of claims 11-18, wherein the anti-cancer therapy comprises Osimertinib, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

70. The method of any one of claims 11-18, wherein the anti-cancer therapy comprises Erlotinib, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

71. The method of any one of claims 11-18, wherein the anti-cancer therapy comprises Gefitinib, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

72. The method of any one of claims 11-18, wherein the anti-cancer therapy comprises Necitumumab, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

73. The method of any one of claims 11-18, wherein the anti-cancer therapy comprises Panitumumab, and the SL partner genes comprise TOMM40L, NTRK1, ALS2, AMMECR1L, B4GALT5, PACS1, CSMD3, KLHL34, AIRE, VPS13B, LCT, CHRFAM7A, PGCP, MTBP, RUFY1, RIMS2, TRAF3, TRIM71, TMEM74, MATN3, C14orf145, NIPAL2, LARP1, LSM11, and CLIP2.

74. The method of any one of claims 11-18, wherein the anti-cancer therapy comprises Trametinib, and the SL partner genes comprise CPT2, RPL5, CTDSPL2, MAP1LC3A, HBS1L, USP3, MRTO4, ELOVL6, PRKACG, TADA2A, CUL5, ME3, STXBP3, DHX35, CSNK1G3, RBBP4, MRE11A, NAA16, BCAT2, RAB33B, DDX46, CABP5, WDR26, LACTB2, and PRPSAP1.

75. The method of any one of claims 11-18, wherein the anti-cancer therapy comprises Temsirolimus, and the SL partner genes comprise PRKCG, DNAJB12, CDK16, C20orf118, STK4, CTH, NOX1, DNAJC2, FGR, TAF12, DDOST, RPS6KA1, TSSK3, RHEBL1, SLC7A6, MYLK4, KMO, PSMB9, HERC4, SGPL1, PTPN6, SLC2A5, NOL10, TRNAU1AP, and VAC14.

76. The method of any one of claims 11-18, wherein the anti-cancer therapy comprises Ramucirumab, and the SL partner genes comprise PIGT, KNTC1, SNX5, LEP, TAX1BP3, FAM83D, ERGIC1, FGF5, LRRC42, MYC, KCNA7, HOXC8, CDC25B, P4HA3, KIF3C, GREM1, CDC25B, CDC25B, LAMB1, FLNC, FHOD1, SLC7A6, ZFHX4, ITGA5, and NUMBL.

77. The method of any one of claims 11-18, wherein the anti-cancer therapy comprises Ribociclib, and the SL partner genes comprise TPT1, RPL34, RPS6KA6, CSNK1G3, ATP13A4, ACSS2, IMMP2L, PMPCB, RDH13, ATP13A5, ADI1, SUCLA2, MAN1C1, ARL6, MAP1LC3A, SLC9A9, EIF3D, PLS1, EIF1, UCP1, ACSS3, AHCYL1, MOBKL2C, RPL4, and RPS20.

78. A method of determining the sensitivity of a cancer of a subject to an anti-cancer agent, wherein the anti-cancer agent is selected from Table 1, the method comprising:
a. providing expression levels of SL partner genes in a sample of the cancer from the subject, wherein the SL partner genes comprise a plurality of genes associated with the anti-cancer therapy in Table 1;
b. providing expression levels of the SL partner genes in each of a plurality of reference cancer samples;
c. counting the number of the SL partner genes from the subject cancer sample that are downregulated compared to expression levels of the respective SL partner genes among the reference cancer samples; and,
d. dividing the number counted in (c) by the total number of the SL partner genes, thereby generating a SL-score;
wherein a SL-score >0.44 indicates that the subject's cancer is sensitive to the anti-cancer therapy.

79. The method of claim 78, wherein the SL partner genes in steps (c) and (d) consist of the genes associated with the anti-cancer therapy in Table 1.

80. The method of claim 78 or claim 79, wherein a SL partner gene in the subject cancer sample is downregulated if the expression level of the SL partner gene in the subject cancer sample is in the bottom tertile of expression levels of the respective SL partner gene among the reference cancer samples.

81. The method of any one of claims 78-80, wherein the subject's cancer and the reference cancer samples are of the same type of cancer.

82. The method of any one of claims 78-81, wherein the SL partner gene expression levels are measured from RNA-sequencing (RNAseq) or microarray data.

83. The method of any one of claims 78-82, wherein the SL partner gene expression levels are normalized.

84. The method of claim 83, wherein the SL partner gene expression levels are measured from RNAseq and the normalization method is Reads per Kilobase per Million mapped reads (RPKM)/RNAseq by Expectation-Maximization (RSEM).

85. The method of any one of claims 78-84, wherein the SL partner gene expression levels of the reference cancer samples are from the Cancer Genome Atlas (TCGA).

86. A method of treating a cancer in a subject in need thereof, comprising administering a PD1/PDL1 inhibitor to the subject, wherein a sample of the cancer has been determined to have a Synthetic Rescue (SR)-score 0.9, wherein the SR-score was determined by:
a. providing expression levels of SR partner genes in the subject cancer sample, wherein the SR partner genes comprise CXCL16, IL15RA, CD27, TNFRSF13C, TNFRSF13B, ICAM4, CD8A, CD4, LTBR, and IFITM2;
b. providing expression levels of the SR partner genes in each of a plurality of reference cancer samples;
c. counting the number of the SR partner genes from the subject cancer sample that are downregulated compared to expression levels of the respective SR partner genes among the reference cancer samples;
d. dividing the number counted in (c) by the total number of the SR partner genes; and,
e. subtracting the result of (d) from 1;
wherein a SR-score ≥0.9 indicates that the subject's cancer is sensitive to the PD1/PDL1 inhibitor.

87. The method of claim 86, wherein the SR partner genes in steps (c) and (d) consist of CXCL16, IL15RA, CD27, TNFRSF13C, TNFRSF13B, ICAM4, CD8A, CD4, LTBR, and IFITM2.

88. The method of claim 86 or claim 87, wherein the SR partner gene in the subject cancer sample is downregulated if the expression level of the SR partner gene in the subject cancer sample is in the bottom tertile of expression levels of the respective SR partner gene among the reference cancer samples.

89. The method of any one of claims 86-88, wherein the subject's cancer and the reference cancer samples are of the same type of cancer.

90. The method of any one of claim 86-89, wherein SR partner gene expression levels are measured from RNAseq or microarray data.

91. The method of any one of claims 86-90, wherein the SR partner gene expression levels are normalized.

92. The method of claim 91, wherein the SR partner gene expression levels are measured from RNAseq and the normalization method is Reads per Kilobase per Million mapped reads (RPKM)/RNAseq by Expectation-Maximization (RSEM).

93. The method of any one of claims 86-92, wherein the SR partner gene expression levels of the reference cancer samples are from the Cancer Genome Atlas (TCGA).

94. The method of any one of claims 86-93, wherein the PD1/PDL1 inhibitor is selected from the group consisting of Pembrolizumab, Nivolumab, Cemiplimab, Atezolizumab, Avelumab, and Durvalumab.

95. A method of treating a cancer in a subject in need thereof, comprising administering an anti-CTLA4 therapy to the subject, wherein a sample of the cancer has been determined to have a Synthetic Rescue (SR)-score ≥0.9, wherein the SR-score was determined by:
a. providing expression levels of SR partner genes in the subject cancer sample, wherein the SR partner genes comprise CD44, IL22RA2, THBD, BID, F12, CCL13, EWSR1, CD274, IL22RA1, and CDKN1A;
b. providing expression levels of the SR partner genes in each of a plurality of reference cancer samples;
c. counting the number of the SR partner genes from the subject cancer sample that are downregulated compared to expression levels of the respective SR partner genes among the reference cancer samples;
d. dividing the number counted in (c) by the total number of the SR partner genes; and,
e. subtracting the result of (d) from 1;
wherein a SR-score ≥0.9 indicates that the subject's cancer is sensitive to the anti-CTLA4 therapy.

96. The method of claim 95, wherein the SR partner genes in steps (c) and (d) consist of CD44, IL22RA2, THBD, BID, F12, CCL13, EWSR1, CD274, IL22RA1, and CDKN1A.

97. The method of claim 95 or claim 96, wherein the SR partner gene in the subject cancer sample is downregulated if the expression level of the SR partner gene in the subject cancer sample is in the bottom tertile of expression levels of the respective SR partner gene among the reference cancer samples.

98. The method of any one of claims 95-97, wherein the subject's cancer and the reference cancer samples are of the same type of cancer.

99. The method of any one of claim 95-98, wherein SR partner gene expression levels are measured from RNAseq or microarray data.

100. The method of any one of claims 95-99, wherein the SR partner gene expression levels are normalized.

101. The method of claim 100, wherein the SR partner gene expression levels are measured from RNAseq and the normalization method is Reads per Kilobase per Million mapped reads (RPKM)/RNAseq by Expectation-Maximization (RSEM).

102. The method of any one of claims 95-101, wherein the SR partner gene expression levels of the reference cancer samples are from the Cancer Genome Atlas (TCGA).

103. The method of any one of claims 95-102, wherein the anti-CTLA4 therapy is selected from the group consisting of Ipilimumab and tremelimumab.

104. A method of determining the sensitivity of a cancer of a subject to a PD1/PDL1 inhibitor, the method comprising:
a. providing expression levels of SR partner genes in a sample of the cancer from the subject, wherein the SR partner genes comprise CXCL16, IL15RA, CD27, TNFRSF13C, TNFRSF13B, ICAM4, CD8A, CD4, LTBR, and IFITM2;
b. providing expression levels of the SR partner genes in each of a plurality of reference cancer samples;
c. counting the number of the SR partner genes from the subject cancer sample that are downregulated compared to expression levels of the respective SR partner genes among the reference cancer samples;
d. dividing the number counted in (c) by the total number of the SR partner genes; and,
e. subtracting the result of (d) from 1, thereby generating a SR-score;
wherein a SR-score ≥0.9 indicates that the subject's cancer is sensitive to the PD1/PDL1 inhibitor.

105. The method of claim 104, wherein the SR partner genes in steps (c) and (d) consist of CXCL16, IL15RA, CD27, TNFRSF13C, TNFRSF13B, ICAM4, CD8A, CD4, LTBR, and IFITM2.

106. The method of claim 104 or claim 105, wherein the SR partner gene in the subject cancer sample is downregulated if the expression level of the SR partner gene in the subject cancer sample is in the bottom tertile of expression levels of the respective SR partner gene among the reference cancer samples.

107. The method of any one of claims 104-106, wherein the subject's cancer and the reference cancer samples are of the same type of cancer.

108. The method of any one of claim 104-107, wherein SR partner gene expression levels are measured from RNAseq or microarray data.

109. The method of any one of claims 104-108, wherein the SR partner gene expression levels are normalized.

110. The method of claim 109, wherein the SR partner gene expression levels are measured from RNAseq and the normalization method is Reads per Kilobase per Million mapped reads (RPKM)/RNAseq by Expectation-Maximization (RSEM).

111. The method of any one of claims 104-110, wherein the SR partner gene expression levels of the reference cancer samples are from the Cancer Genome Atlas (TCGA).

112. The method of any one of claims 104-111, wherein the PD1/PDL1 inhibitor is selected from the group consisting of Pembrolizumab, Nivolumab, Cemiplimab, Atezolizumab, Avelumab, and Durvalumab.

113. A method of determining the sensitivity of a cancer of a subject to an anti-CTLA4 therapy, the method comprising:
a. providing expression levels of SR partner genes in a sample of the cancer from the subject, wherein the SR partner genes comprise CD44, IL22RA2, THBD, BID, F12, CCL13, EWSR1, CD274, IL22RA1, and CDKN1A;
b. providing expression levels of the SR partner genes in each of a plurality of reference cancer samples;
c. counting the number of the SR partner genes from the subject cancer sample that are downregulated compared to expression levels of the respective SR partner genes among the reference cancer samples;
d. dividing the number counted in (c) by the total number of the SR partner genes; and,
e. subtracting the result of (d) from 1, thereby generating a SR-score;
wherein a SR-score ≥0.9 indicates that the subject's cancer is sensitive to the anti-CTLA4 therapy.

114. The method of claim 113, wherein the SR partner genes in steps (c) and (d) consist of CD44, IL22RA2, THBD, BID, F12, CCL13, EWSR1, CD274, IL22RA1, and CDKN1A.

115. The method of claim 113 or claim 114, wherein the SR partner gene in the subject cancer sample is downregulated if the expression level of the SR partner gene in the subject cancer sample is in the bottom tertile of expression levels of the respective SR partner gene among the reference cancer samples.

116. The method of any one of claims 113-115, wherein the subject's cancer and the reference cancer samples are of the same type of cancer.

117. The method of any one of claim 113-116, wherein SR partner gene expression levels are measured from RNAseq or microarray data.

118. The method of any one of claims 113-117, wherein the SR partner gene expression levels are normalized.

119. The method of claim 113-118, wherein the SR partner gene expression levels are measured from RNAseq and the normalization method is Reads per Kilobase per Million mapped reads (RPKM)/RNAseq by Expectation-Maximization (RSEM).

120. The method of any one of claims 113-119, wherein the SR partner gene expression levels of the reference cancer samples are from the Cancer Genome Atlas (TCGA).

121. The method of any one of claims 113-120, wherein the anti-CTLA4 therapy is selected from the group consisting of Ipilimumab and tremelimumab.
